(19) United States
(12) Patent Application Publication
(10) Pub. No.: US 2023/0326253 A1
(43) Pub. Date: Oct. 12, 2023

(54) BIOMETRIC AUTHENTICATION SYSTEM AND BIOMETRIC AUTHENTICATION METHOD

(71) Applicant: Panasonic Intellectual Property Management Co., Ltd., Osaka (JP)

(72) Inventors: SANSHIRO SHISHIDO, Osaka (JP); SHINICHI MACHIDA, Osaka (JP)

(21) Appl. No.: 18/327,931

(22) Filed: Jun. 2, 2023

Related U.S. Application Data
(63) Continuation of application No. PCT/JP2021/044433, filed on Dec. 3, 2021.

(30) Foreign Application Priority Data
Dec. 23, 2020 (JP) 2020-214155

(51) Int. Cl. H04N 23/56; H04N 23/11; G06V 10/143; H04N 23/13; G06V 40/40; G06V 40/16

(52) U.S. Cl. CPC G06V 40/45; G06V 10/143; G06V 40/16; H04N 23/11; H04N 23/13; H04N 23/56

(57) ABSTRACT

A biometric authentication system includes a first image capturer that captures a visible light image that is imaged by picking up first light reflected from a skin portion of a subject that is irradiated with visible light; a second image capturer that captures a first infrared image that is imaged by picking up second light that is reflected from the skin portion irradiated with first infrared light and that has a wavelength region including a first wavelength; and a determiner that determines, in accordance with a result of comparing the visible light image with the first infrared image, whether the subject is a living body and outputs a determination result.

### BACKGROUND

### 1. Technical Field

The present disclosure relates to a biometric authentication system and a biometric authentication method.

### 2. Description of the Related Art

The importance of personal authentication method using biometric authentication is increasing. For example, the personal authentication may be applied to office entrance/exit management, immigration control, transactions in financial institutions or transaction using smart phones, and public monitoring cameras. Authentication accuracy of the personal authentication is increased using machine learning together with a vast amount of database and improved algorithms. On the other hand, the problem of impersonation arises in the personal authentication using the biometric authentication. For example, Japanese Unexamined Patent Application Publication No. 2017-228316 discloses a detector that detects a disguise item used to impersonate.

In the biometric authentication, there is a demand for authentication accuracy coping with impersonation and miniaturization of a biometric authentication device.

### SUMMARY

In one general aspect, the techniques disclosed here feature a biometric authentication system including a first image capturer that captures a visible light image that is imaged by picking up first light reflected from a skin portion of a subject that is irradiated with visible light; a second image capturer that captures a first infrared image that is imaged by picking up second light that is reflected from the skin portion irradiated with first infrared light and that has a wavelength region including a first wavelength; and a determiner that determines, in accordance with a result of comparing the visible light image with the first infrared image, whether the subject is a living body and outputs a determination result.

It should be noted that general or specific embodiments may be implemented as a system, a method, an integrated circuit, a computer program, a storage medium, or any selective combination thereof.

Additional benefits and advantages of the disclosed embodiments will become apparent from the specification and drawings. The benefits and/or advantages may be individually obtained by the various embodiments and features of the specification and drawings, which need not all be provided in order to obtain one or more of such benefits and/or advantages.

### BRIEF DESCRIPTION OF THE DRAWINGS

FIG. 1 illustrates a process of a biometric authentication system of a first embodiment that performs impersonation determination;

FIG. 2 is a block diagram illustrating a functional configuration of the biometric authentication system of the first embodiment;

FIG. 3 illustrates an example of a visible light image and a first infrared image that are comparison targets that a determiner of the first embodiment compares;

FIG. 4 schematically illustrates light reflectance properties of a living body;

FIG. 5 illustrates an example of a reflection ratio of visible light incident on the skin of human;

FIG. 6 illustrates an nk spectrum of liquid water;

FIG. 7 illustrates images that are imaged by photographing a human face on different waveforms;

FIG. 8 illustrates a waveform dependency of reflectance of light responsive to the color of skin;

FIG. 9 illustrates sunlight spectrum on the ground;

FIG. 10 illustrates in enlargement a portion of the sunlight spectrum in FIG. 9;

FIG. 11 illustrates in enlargement another portion of the sunlight spectrum in FIG. 9;

FIG. 12 is a flowchart illustrating a process example of the biometric authentication system of the first embodiment;

FIG. 13 illustrates a process of the biometric authentication system of the first embodiment that performs the impersonation determination when a subject is not impersonated;

FIG. 14 is a block diagram illustrating a functional configuration of a biometric authentication system according to a modification of the first embodiment;

FIG. 15 illustrates a configuration example of a third imaging device according to the modification of the first embodiment;

FIG. 16 is a schematic cross-sectional view illustrating a cross-sectional structure of a pixel of the third imaging device according to the modification of the first embodiment;

FIG. 17 schematically illustrates an example of a spectral sensitivity curve of a pixel according to the modification of the first embodiment;

FIG. 18 is a schematic cross-sectional view illustrating a cross-sectional structure of another pixel of the third imaging device according to the modification of the first embodiment;

FIG. 19 is a schematic cross-sectional view illustrating a cross-sectional structure of another pixel of the third imaging device according to the modification of the first embodiment;

FIG. 20 schematically illustrates an example of spectral sensitivity curves of another pixel according to the modification of the first embodiment;

FIG. 21 is a block diagram illustrating a functional configuration of a biometric authentication system of a second embodiment;

FIG. 22 is a flowchart illustrating a process example of the biometric authentication system of the second embodiment;

FIG. 23 is a block diagram illustrating a functional configuration of a biometric authentication system according to a modification of the second embodiment;

FIG. 24 is a schematic cross-sectional view illustrating a cross-sectional structure of a pixel of a fifth imaging device according to the modification of the second embodiment; and

FIG. 25 schematically illustrates an example of spectral sensitivity curves of a pixel according to the modification of the second embodiment.

### DETAILED DESCRIPTIONS

### Underlying Knowledge Forming Basis of the Present Disclosure

With the vast amount of image database globally available or individually acquired and the advancement of machine learning algorithms, authentication rate is improved in biometric authentication, such as face recognition, using a visible light image.

A problem of unauthorized authentication, such as a third party impersonating an authentic user, arises in biometric authentication based on images resulting from photographing subjects. For example, the third party may impersonate an authentic user using a printed image of the authentic user, an image of the authentic user displayed on a terminal, such as a smart phone or a tablet, or a three-dimensional mask manufactured of paper, silicone, or rubber.

Japanese Unexamined Patent Application Publication No. 2017-228316 discloses a technique of detecting impersonation by using multiple infrared images that are imaged by photographing a subject irradiated with infrared rays mutually different wavelength regions. According to the technique, however, two problems arise. A first problem is that the user of the infrared image reduces the authentication rate in personal authentication because of an insufficient amount of database. A second problem is that the use of multiple infrared wavelength regions leads to an increase in the number of imagers, an addition of spectroscopy system and light source, and an increase in the amount of image data to be processed.

As described below, the inventors have found that the impersonation determination that determines in accordance with a visible light image and an infrared image whether a subject is impersonated leads to downsizing an apparatus in use rather than enlarging the apparatus, and a higher accuracy level of the biometric authentication in the impersonation determination and personal authentication.

### Outline of Disclosure

Aspects of the disclosure are described below.

A biometric authentication system according to an aspect of the disclosure includes:
a first image capturer that captures a visible light image that is imaged by picking up first light reflected from a skin portion of a subject that is irradiated with visible light;
a second image capturer that captures a first infrared image that is imaged by picking up second light that is reflected from the skin portion irradiated with first infrared light and that has a wavelength region including a first wavelength; and
a determiner that determines, in accordance with a result of comparing the visible light image with the first infrared image, whether the subject is a living body and outputs a determination result.

If the subject is a living body, part of infrared light entering the living body is absorbed by a water component in a surface region of the living body and the first infrared image has a portion darker than the visible light image. Simply comparing the two types of images, namely, the visible light image and first infrared image, may easily help determine whether the subject is a living body, or an artificial object used to impersonate, such as a screen on a terminal, paper, silicone rubber, or the like. The biometric authentication system may thus be downsized. Regardless of the shape of a subject, namely, whether the subject for impersonation has a planar shape or a three-dimensional shape, a difference in darkness occurs between the visible light image and first infrared image and the impersonation determination may be performed at a higher accuracy level. According to the disclosure, the biometric authentication system may have a higher accuracy authentication and be downsized.

The biometric authentication system may include a first authenticator that performs first personal authentication on the subject in accordance with the visible light image and that outputs a result of the first personal authentication.

The first authenticator performs personal authentication on the subject in accordance with the visible light image, leading to a sufficiently available database of visible light images. The biometric authentication system thus enables personal authentication to be at a higher accuracy level.

If the determiner determines that the subject is not the living body, the first authenticator may not perform the first personal authentication on the subject.

Processing workload in the biometric authentication system may thus be reduced.

The biometric authentication system may further include a second authenticator that performs second personal authentication on the subject in accordance with the first infrared image and that outputs a result of the second personal authentication.

Since a ratio of a surface reflection component to a diffuse reflection component in infrared light reflected from the living body irradiated with infrared light is higher than a ratio of a surface reflection component to a diffuse reflection component in visible light reflected from the living body irradiated with visible light, the first infrared image is higher in spatial resolution than the visible light image. For this reason, in addition to the personal authentication performed by the first authenticator, the second authenticator performs biometric authentication in accordance with the first infrared image having a higher spatial resolution. A higher accuracy personal authentication may thus result.

The biometric authentication system may further include:
a storage that stores information used to perform the first personal authentication and the second personal authentication; and
an information constructor that causes the storage to store information on the result of the first personal authentication and information on the result of the second personal authentication in an associated form.

Database of the first infrared images higher in spatial resolution than the visible light images but smaller in amount than the visible light images may thus be expanded. The biometric authentication system enabled to perform higher-accuracy personal authentication may thus implemented by performing machine learning using the database.

The determiner may compare a contrast value based on the visible light image with a contrast value based on the first infrared image to determine whether the subject is the living body.

The biometric authentication system may thus perform the impersonation determination using the contrast values that are easy to calculate.

The biometric authentication system may further include an imager that includes a first imaging device imaging the visible light image and a second imaging device imaging the first infrared image,
the first image capturer may capture the visible light image from the first imaging device, and
the second image capturer may capture the first infrared image from the second imaging device.

Since the visible light image and first infrared image are respectively imaged by the first imaging device and second imaging device, the biometric authentication system may be implemented by using simple-structured cameras in the first imaging device and the second imaging device.

The biometric authentication system may further include an imager that includes a third imaging device imaging the visible light image and the first infrared image,
the first image capturer may capture the visible light image from the third imaging device, and
the second image capturer may capture the first infrared image from the third imaging device.

Since the third imaging device images both the visible light image and the first infrared image, the biometric authentication system may be even more downsized.

The third imaging device may include a first photoelectric conversion layer having a spectral sensitivity to a wavelength range of the visible light and the first wavelength.

The third imaging device that images the visible light image and the first infrared image is implemented using one photoelectric conversion layer. Manufacturing of the third imaging device may thus be simplified.

The third imaging device may include a second photoelectric conversion layer having a spectral sensitivity to an entire wavelength range of visible light.

The use of the second photoelectric conversion layer may improve the image quality of the visible light image, thereby increasing the accuracy of the biometric authentication based on the visible light image.

The biometric authentication system may further include a light illuminator that irradiates the subject with the first infrared light.

Since the subject is irradiated with infrared light by an active light illuminator, the image quality of the first infrared image picked up by the second imaging device may be improved, and the authentication accuracy of the biometric authentication system may be increased.

The biometric authentication system may further include a timing controller that controls an imaging timing of the imager and an irradiation timing of the light illuminator.

Since the subject is irradiated with infrared light only for a time of duration for the biometric authentication, power consumption may be reduced.

The biometric authentication system may further include a third image capturer that captures a second infrared image that is imaged by picking up third light that is reflected from the skin portion irradiated with second infrared light and that has a wavelength region including a second wavelength different from the first wavelength; and

the determiner may determine in accordance with the visible light image, the first infrared image, and the second infrared image whether the subject is the living body.

The determiner determines whether the subject is the living body by using the second infrared image that is imaged by picking up infrared light different in wavelength from the first infrared image. The determination accuracy of the determiner may thus be increased.

The determiner may generate a difference infrared image between the first infrared image and the second infrared image and may determine, in accordance with the difference infrared image and the visible light image, whether the subject is the living body.

An image resulting from picking up infrared light may have a determination difficulty in response to the absorption of irradiation light by the water component or the shadow of the irradiation light. The difference infrared image between the first infrared image and the second infrared image different in wavelength is generated. The use of the difference infrared image removes the effect caused when the dark portion results from the shadow of the irradiation light. The authentication accuracy of the biometric authentication system may thus be increased.

The first wavelength may be shorter than or equal to 1,100 nm.

This arrangement may implement a biometric authentication system including an imager employing a low-cost silicon sensor.

The first wavelength may be longer than or equal to 1,200 nm.

This arrangement leads to larger absorption of infrared light by the water component of the living body, creating a clear contrast of the first infrared image, and increasing the authentication accuracy of the biometric authentication system.

The first wavelength may be longer than or equal to 1,350 nm and shorter than or equal to 1,450 nm.

The wavelength range longer than or equal to 1,350 nm and shorter than or equal to 1,450 nm is a missing wavelength range of the sunlight and has a higher absorption coefficient by the water component. The wavelength range is thus less influenced by ambient light and leads to a clearer contrast of the first infrared image. The authentication accuracy of the biometric authentication system may thus be increased.

The subject may be a human face.

The biometric authentication system performing face recognition may thus have higher authentication accuracy and may be downsized.

A biometric authentication method according to an aspect of the disclosure includes:
capturing a visible light image that is imaged by picking up first light reflected from a skin portion of a subject that is irradiated with visible light;
capturing a first infrared image that is imaged by picking up second light that is reflected from the skin portion irradiated with first infrared light and that has a wavelength region including a first wavelength; and
determining, in accordance with a result of comparing the visible light image with the first infrared image, whether the subject is a living body and outputting a determination result.

In the same way as with the biometric authentication system, the biometric authentication method may easily perform the impersonation determination at a higher accuracy level by simply comparing the visible light image with the first infrared image. According to the disclosure, the biometric authentication method may help downsize a biometric authentication apparatus that performs the biometric authentication method and provides higher accuracy authentication.

An biometric authentication system according to an aspect of the disclosure comprises:
a memory; and
circuitry that, in operation,
retrieves from the memory a visible light image that is imaged by picking up first light reflected from a skip portion of a subject that is irradiated with visible light;
retrieves from the memory a first infrared image that is imaged by picking up second light that is reflected from the skin portion irradiated with first infrared light and has a wavelength region including a first wavelength; and
determines, in accordance with a result of comparing the visible light image with the first infrared image, whether the subject is a living body; and
outputs a determination result.

The circuitry may perform, in operation, first personal authentication on the subject in accordance with the visible light image and output a result of the first personal authentication.

If the circuitry determines that the subject is not a living body, the circuitry may not perform the first personal authentication on the subject.

The circuitry may perform, in operation, second personal authentication on the subject in accordance with the first infrared image and output a result of the second personal authentication.

The biometric authentication system may further include a storage that stores information used to perform the first personal authentication and the second personal authentication,

wherein the circuitry may store information on the result of the first personal authentication and information on the result of the second personal authentication in association with each other.

The circuitry may determine whether the subject is a living body, by comparing a contrast value based on the visible light image and a contrast value based on the first infrared image.

The circuity may further control, in operation, an imaging timing of the imager and an irradiation timing of the light illuminator.

The biometric authentication system may further include a third image capturer that captures a second infrared image that is imaged by picking up third light that is reflected from the skin portion irradiated with second infrared light and that has a wavelength region including a second wavelength different from the first wavelength; and

wherein the circuitry may determine in accordance with the visible light image, the first infrared image, and the second infrared image whether the subject is the living body.

The circuitry may generate a difference infrared image from between the first infrared image and the second infrared image and determine, in accordance with the difference infrared image and the visible light image, whether the subject is the living body.

According to the disclosure, a circuit, a unit, an apparatus, an element, a portion of the element, and all or a subset of functional blocks in a block diagram may be implemented by one or more electronic circuits including a semiconductor device, a semiconductor integrated circuit (IC), or a large-scale integrated (LSI) circuit. The LSI or IC may be integrated into a single chip or multiple chips. For example, a functional block other than a memory element may be integrated into a single chip. The LSI and IC are quoted herein. Depending on the degree of integration, integrated circuits may be also referred to as a system LSI, a very large-scale integrated (VLSI) circuit, or an ultra-large-scale integrated (ULSI) circuit and these circuits may also be used. Field programmable gate array (FPGA) that is programmed on an LSI after manufacturing the LSI may also be employed. Reconfigurable logic device permitting a connection in an LSI to be reconfigured or permitting a circuit region in an LSI to be set up may also be employed.

The function or operation of the circuit, the unit, the apparatus, the element, the portion of the element, and all or a subset of functional blocks may be implemented by a software program. In such a case, the software program may be stored on a non-transitory recording medium, such as one or more read-only memories (ROMs), an optical disk, or a hard disk. When the software program is executed by a processor, the function identified by the software program is thus performed by the processor or a peripheral device thereof. A system or an apparatus may include one or more non-transitory recording media, a processor, and a hardware device, such as an interface.

Embodiments of the disclosure are described in detail by referring to the drawings.

The embodiments described below are general or specific examples. Numerical values, shapes, elements, layout locations and connection configurations of the elements, steps, orders of the steps are recited for exemplary purposes only and do not intend to limit the disclosure. From among the elements in the embodiments, an element not recited in an independent claim may be construed as an optional element. The drawings are not necessarily drawn to scale. For example, in each drawing, scale is not necessarily consistent. In the drawings, elements substantially identical in configuration are designated with the same reference symbol and the discussion thereof is simplified or not repeated.

According to the specification, a term representing a relationship between elements, a term representing the shape of each element, and a range of each numerical value are used not only in a strict sense but also in a substantially identical sense. For example, this allows a tolerance of few percent with respect to a quoted value.

In the specification, the terms “above” and “below” are not used to specify a vertically upward direction or a vertically downward direction in absolute spatial perception but may define a relative positional relationship based on the order of lamination in a layer structure. Specifically, a light incident side of an imaging device may be referred to as “above” and an opposite side of the light incident side may be referred to as “below.” The terms “above” and “below” are simply used to define a layout location of members and does not intend the posture of the imaging device in use. The terms “above” and “below” are used when two elements are mounted with space therebetween such that another element is inserted in the space or when the two elements are mounted in contact with each other with no space therebetween.

### First Embodiment

### Outline

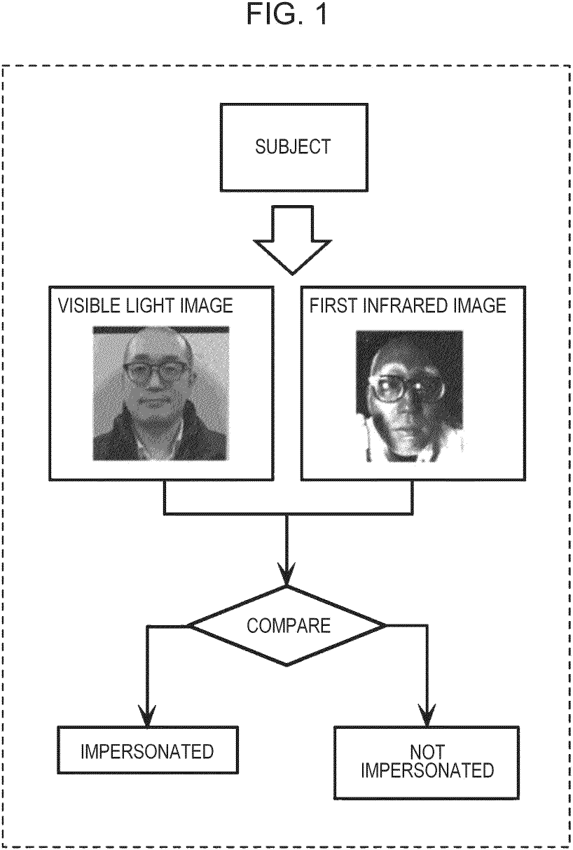

The outline of a biometric authentication process of a biometric authentication system of a first embodiment is described. The biometric authentication system of the first embodiment performs, in biometric authentication, impersonation determination about a subject, and personal authentication of the subject. In the context of the specification, each of the impersonation determination and the personal authentication is an example of the biometric authentication. FIG. 1 schematically illustrates the impersonation determination of the biometric authentication system of the first embodiment.

Referring to FIG. 1, the biometric authentication system of the first embodiment compares a visible light image that is imaged by picking up visible light with a first infrared image that is imaged by picking up infrared light. Through the comparison, the biometric authentication system determines whether the subject is (i) a living body and thus not impersonated or (ii) an artificial object imitating a living body and thus impersonated. According to the first embodiment, the wavelength range of visible light is longer than or equal to 380 nm and shorter than 780 nm. The wavelength range of infrared light is longer than or equal to 780 nm and shorter than or equal to 4,000 nm. In particular, shortwave infrared (SWIR) having a wavelength range of longer than or equal to 900 nm and shorter than or equal to 2,500 nm is used as the infrared light. In the specification, electromagnetic waves including visible light and infrared light are simply referred to as “light” for convenience of explanation.

The subject serving as a target of the biometric authentication is, for example, a human face. The subject is not limited to the human face, and may be a portion of the living body other than the human face. For example, the subject may be a portion of a hand of the human, such as a finger print or a palm print. The subject may be the entire body of the human.

Related-art impersonation determination methods using infrared light include spectroscopic method that acquires multiple infrared light wavelengths and an authentication method that acquires three-dimensional data by distance measurement. The spectroscopic method involves an increase in system scale and the authentication method is unable to determine impersonation using a three-dimensional structure manufactured of paper or silicone rubber. In view of recent performance improvement of three-dimensional printers, the impersonation determination based on shape recognition alone is becoming more difficult in the biometric authentication using a face, finger print, or palm print. In contrast, as illustrated in FIG. 1, the impersonation determination of the first embodiment is performed in accordance with a change that takes place in the difference between the visible light image and the first infrared image depending on a living body or an artificial object. A higher-accuracy biometric authentication may be performed by simply acquiring the two images without increasing apparatus scale.

### Configuration

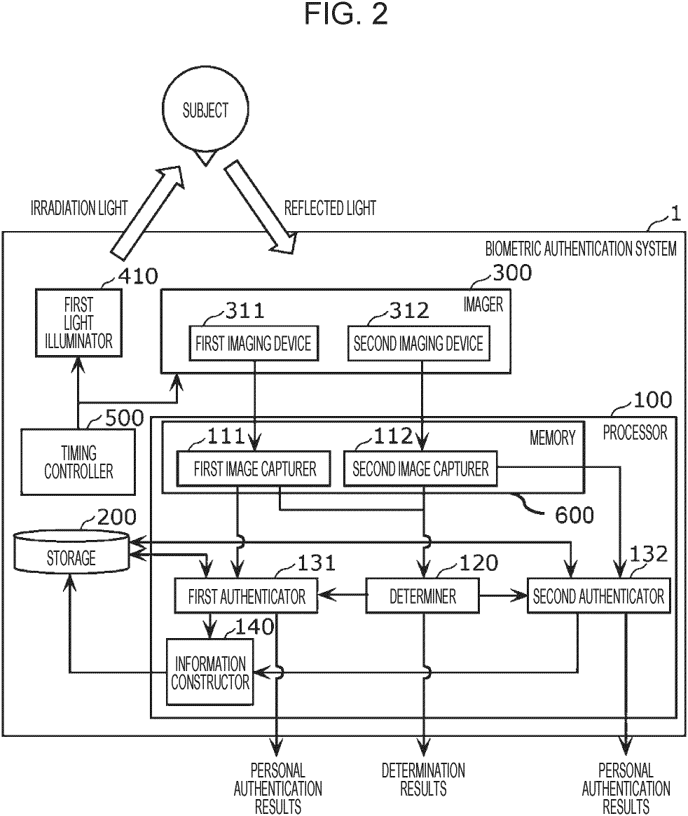

Configuration of the biometric authentication system of the first embodiment is described below. FIG. 2 is a functional block diagram illustrating a biometric authentication system 1 of the first embodiment.

Referring to FIG. 2, the biometric authentication system 1 includes a processor 100, a storage 200, an imager 300, a first light illuminator 410, and a timing controller 500. The first light illuminator 410 is an example of a light illuminator.

The processor 100 is described herein in greater detail. The processor 100 in the biometric authentication system 1 performs an information processing process, such as impersonation determination and personal authentication. The processor 100 includes a memory 600, including a first image capturer 111 and a second image capturer 112, a determiner 120, a first authenticator 131, a second authenticator 132, and an information constructor 140. The processor 100 may be implemented by a microcontroller including one or more processors storing programs. The function of the processor 100 may implemented by a combination of a general-purpose processing circuit and a software component or by a hardware component that is specialized in the process of the processor 100.

The first image capturer 111 captures a visible light image of a subject. The first image capturer 111 temporarily stores the visible light image of the subject. The visible light image is imaged by picking up light reflected from the subject irradiated with visible light. The first image capturer 111 captures the visible light image from the imager 300, specifically, a first imaging device 311 in the imager 300. The visible light image is a color image including information on a luminance value of each of red (R), green (G), and blue (B) colors. The visible light image may be a grayscale image.

The second image capturer 112 captures the first infrared image of the subject. The second image capturer 112 temporarily stores the first infrared image of the subject. The first infrared image is imaged by picking up light that is reflected from the subject irradiated with infrared light and includes a wavelength region including a first wavelength. The second image capturer 112 captures the first infrared image from the imager 300, specifically, from a second imaging device 312 in the imager 300.

In response to the visible light image captured by the first image capturer 111 and the first infrared image captured by the second image capturer 112, the determiner 120 determines whether the subject is a living body. The determiner 120 determines whether the subject is a living body, by comparing a contrast value of the visible light image with a contrast value of the first infrared image. A detailed process performed by the determiner 120 is described below.

The determiner 120 outputs determination results as a determination signal to the outside. The determiner 120 may also output the determination results as the determination signal to the first authenticator 131 and the second authenticator 132.

The first authenticator 131 performs personal authentication on the subject in accordance with the visible light image captured by the first image capturer 111. For example, if the determiner 120 determines that the subject is not a living body, the first authenticator 131 does not perform the personal authentication on the subject. The first authenticator 131 outputs results of the personal authentication to the outside.

The second authenticator 132 preforms the personal authentication on the subject in response to the first infrared image captured by the second image capturer 112. The second authenticator 132 outputs results of the personal authentication to the outside.

The information constructor 140 stores in an associated form on the storage 200 information on the results of the personal authentication performed by the first authenticator 131 and information on the results of the personal authentication performed by the second authenticator 132. For example, the information constructor 140 stores the visible light image and the first infrared image, used in the personal authentication, and the results of the personal authentication on the storage 200.

The storage 200 stores information used to perform the personal authentication. For example, the storage 200 stores a personal authentication database that associates personal information on the subject with the image depicting the subject. The storage 200 is implemented by, for example, a hard disk drive (HDD). The storage 200 may also be implemented by a semiconductor memory.

The imager 300 images an image used in the biometric authentication system 1. The imager 300 includes the first imaging device 311 and the second imaging device 312.

The first imaging device 311 images the visible light image of the subject. Visible light reflected from the subject irradiated with visible light is incident on the first imaging device 311. The first imaging device 311 generates the visible light image by imaging the incident reflected light. The first imaging device 311 outputs the acquired visible light image. For example, the first imaging device 311 may include an image sensor, a control circuit, a lens, and the like. The image sensor is a charge-coupled device (CCD) or a complementary metal oxide semiconductor (CMOS) sensor, having a spectral sensitivity to visible light. The first imaging device 311 may be a related-art visible-light camera. The first imaging device 311 operates in a global-shutter method in which exposure periods of multiple pixels are unified.

The second imaging device 312 images the first infrared image of the subject. Infrared light reflected from the subject irradiated with infrared light and having a wavelength region including a first wavelength is incident on the second imaging device 312. The second imaging device 312 generates the first infrared image by imaging the incident reflected light. The second imaging device 312 outputs the acquired first infrared image. For example, the second imaging device 312 may include an image sensor, a control circuit, a lens, and the like. The image sensor is a CCD or a CMOS sensor, having a spectral sensitivity to infrared light. The second imaging device 312 may be a related-art infrared-light camera. The second imaging device 312 operates in a global-shutter method in which exposure periods of multiple pixels are unified.

The first light illuminator 410 irradiates the subject with irradiation light that is infrared light within the wavelength range including the first wavelength. The second imaging device 312 images infrared light reflected from the subject that is irradiated with infrared light by the first light illuminator 410. For example, the first light illuminator 410 irradiates the subject with the infrared light having an emission peak on or close to the first wavelength. The use of the first light illuminator 410 may improve the image quality of the first infrared image imaged by the second imaging device 312, leading to an increase in the authentication accuracy of the biometric authentication system 1.

The first light illuminator 410 includes, for example, a light source, a light emission circuit, a control circuit, and the like. The light source used in the first light illuminator 410 is not limited to any type and may be selected according to the purpose of use. For example, the light source in the first light illuminator 410 may be a halogen light source, a light emitting diode (LED) light source, or a laser diode light source. For example, the halogen light source may be used to provide infrared light within a wide range of wavelength. The LED light source may be used to reduce power consumption and heat generation. The laser diode light source may be used when a narrow range of wavelength with the missing wavelength of the sunlight is used or when an authentication rate is increased by using the biometric authentication system 1 together with a distance measurement system.

The first light illuminator 410 may operate not only within a wavelength range including the first wavelength but also within a wavelength range of visible light. The biometric authentication system 1 may further include a lighting device that emits visible light.

The timing controller 500 controls an imaging timing of the imager 300 and an irradiation timing of the first light illuminator 410. For example, the timing controller 500 outputs a first synchronization signal to the second imaging device 312 and the first light illuminator 410. The second imaging device 312 images the first infrared image at the timing responsive to the first synchronization signal. The first light illuminator 410 irradiates the subject with infrared light at the timing responsive to the first synchronization signal. The second imaging device 312 is thus caused to image the subject while the first light illuminator 410 irradiates the subject with infrared light. Since the subject is irradiated with infrared light only for the duration of time for biometric authentication, power consumption may be reduced.

The second imaging device 312 may perform a global shutter operation at a timing responsive to the first synchronization signal. In this way, a motion blur of the subject irradiated with light may be controlled in the resulting image and a higher authentication accuracy may result in the biometric authentication system 1.

The timing controller 500 may be implemented by a microcontroller including one or more processors storing a program. The function of the timing controller 500 may be implemented by a combination of a general-purpose processing circuit and a software component or by a hardware component that is specialized in the process of the timing controller 500.

The timing controller 500 may include an input receiver that receives from a user an instruction to output the first synchronization signal. The input receiver may include a touch panel or physical buttons.

The biometric authentication system 1 may not necessarily include the timing controller 500. For example, the user may directly operate the imager 300 and the first light illuminator 410. The first light illuminator 410 may be continuously on while the biometric authentication system 1 is in use.

### Principle

The principle that the determiner 120 is able to determine in response to the visible light image and the first infrared image whether the subject is a living body is described below.

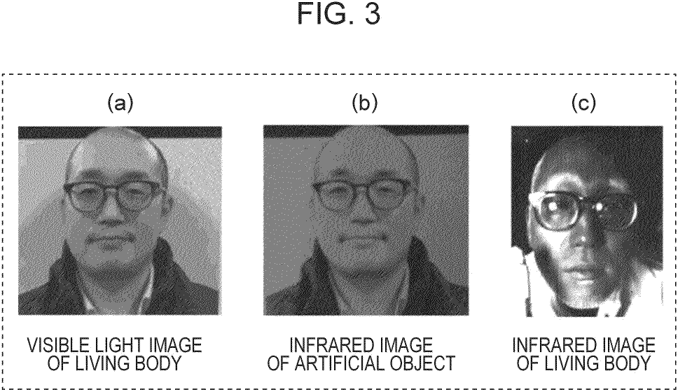

The visible light image and the first infrared image serving as comparison targets on the determiner 120 are described. FIG. 3 illustrates an example of the visible light image and the first infrared image serving as comparison targets on the determiner 120. Part (a) of FIG. 3 is an image of a human face directly taken by a visible-light camera. Specifically, part (a) of FIG. 3 is the visible light image of the subject that is a living body. Part (b) of FIG. 3 is an image taken by an infrared camera that photographs a screen on which the image of the human face is displayed. Specifically, part (b) of FIG. 3 is the first infrared image in which the subject is impersonated with an artificial object. Part (c) of FIG. 3 is an image taken by the infrared camera that directly photographs the human face. Specifically, part (c) of FIG. 3 is the first infrared image of the subject that is a living body. The infrared camera may have a spectral sensitivity to 1,450 nm. The infrared camera includes a bandpass filter that allows light in a wavelength range in the vicinity of 1,450 nm to transmit therethrough. The infrared camera photographs the human face using a light illuminator. The light illuminator includes an LED light source and irradiates the human face with light having a center wavelength of 1,450 nm. The image in part (a) of FIG. 3 is actually a color image but is illustrated as a monochrome image for convenience of explanation.

In the first infrared image with the subject being a living body in part (c) of FIG. 3, skin is darkened by the effect of the absorption by the water component. If the first infrared image in part (c) of FIG. 3 is compared with the visible light image with the subject being a living body in part (a) of FIG. 3, there is a larger difference in contrast and luminance between the first infrared image and the visible light image. On the other hand, if the first infrared image with the subject being impersonated as illustrated in part (b) of FIG. 3 is compared with the visible light image in part (a) of FIG. 3, there is a smaller difference in contrast and luminance between the first infrared image and the visible light image. For example, the contrast value of the first infrared image is larger when the subject is a living body than when the subject is an artificial object. The comparison of these images may facilitate the impersonation determination as to whether the subject is a living body or an artificial object.

The principle that the difference in the contrast illustrated in FIG. 3 is created between the visible light image and the first infrared image is described in greater detail below.

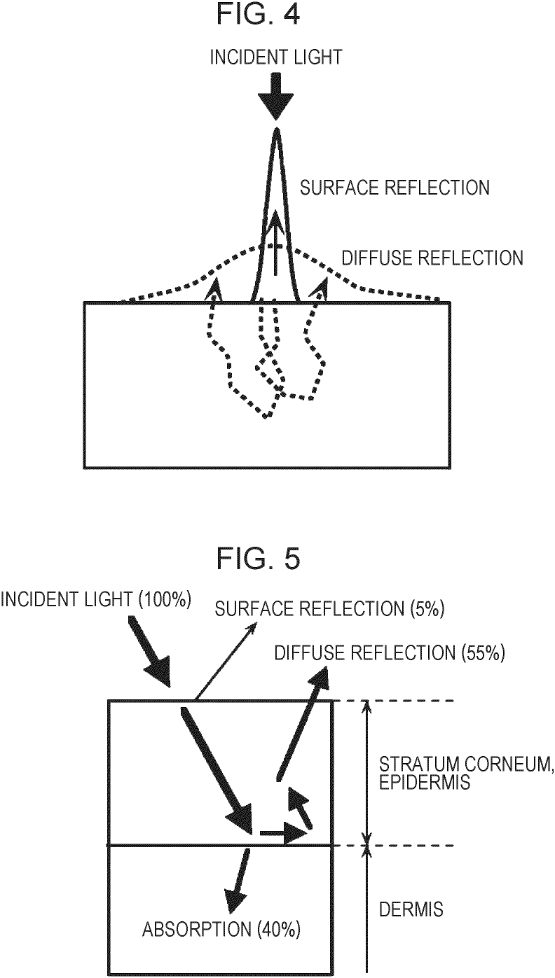

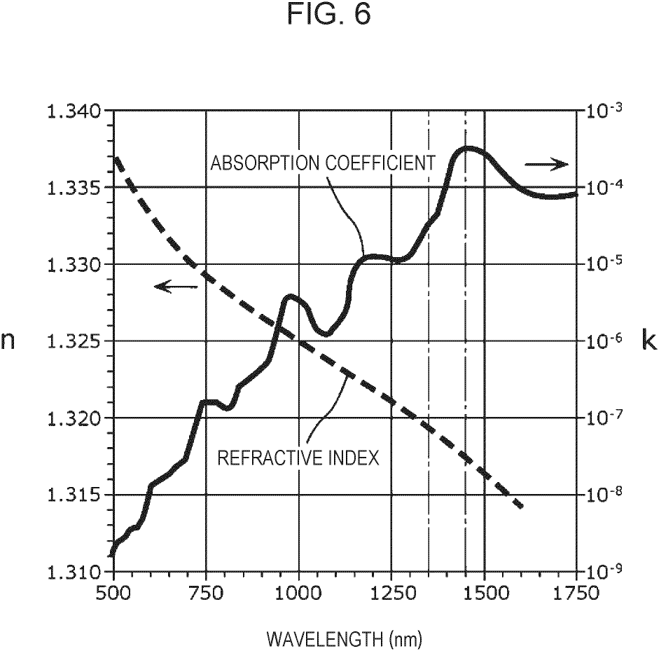

FIG. 4 schematically illustrates light reflectance properties of a living body. Referring to FIG. 4, light is incident on the human skin. FIG. 5 illustrates an example of a reflection ratio of visible light incident on the human skin. FIG. 6 illustrates an nk spectrum of liquid water. Specifically, FIG. 6 illustrates how refractive index n of liquid water and absorption coefficient k by liquid water depend on wavelength of light.

Referring to FIG. 4, light reflected in response to light incident on the human skin is separated into a surface reflection component from the surface of the skin and a diffuse reflectance component that comes out from the skin as a result when light is entered and diffused in subcutaneous tissue. The ratios of the components, such as the surface reflection component, may be illustrated in FIG. 5. When 100% of light is incident on the living body, the surface reflection component is about 5% and the diffuse reflectance component is about 55%. The remaining 40% of the incident light is thermally absorbed by the human dermis and is thus not reflected. If imaging is performed within the visible light wavelength region, about 60% of the overall incident light that is a sum of the surface reflection component and the diffuse reflectance component is thus observed as the reflected light.

Referring to FIG. 6, infrared light in short wave infrared (SWIR) range located on or close to 1,400 nm is higher in absorption coefficient than visible light, and is pronounced in the absorption by the water component. The diffuse reflectance component of infrared light in FIG. 4 is smaller because of the absorption by a water component of the skin, thereby allowing the surface reflection component to be dominant. With reference to the ratios in FIG. 5, the diffuse reflectance component is smaller, thereby allowing the surface reflection component as 5% of the incident light to be observed as the reflected light. For this reason, if the light reflected from the living body in response to infrared light is imaged, a resulting image of the subject appears darker. The comparison of the visible light image and the first infrared image may easily determine whether the subject is a living body or an artificial object. The first embodiment focuses on light reflection properties of the living body that are different depending on the visible light and infrared light, and in particular, focuses on a change in the ratio between the surface reflection component and the diffuse reflectance component in the visible light and infrared light. Since an artificial object, such as a display, paper, or silicone rubber, used to impersonate contains little water component, there occurs no such change in the ratios between the visible light and infrared light attributed to a change in wavelength. For this reason, the visible light image and the first infrared image in FIG. 3 are obtained and compared, allowing the impersonation determination to be performed.

The following ratios are calculated using data on the nk spectrum in FIG. 6. On 550 nm, specular light (namely, the surface reflection component) is about ⅒ the diffuse reflectance component. If the ratio of the diffuse reflectance component is calculated using an average path length of the diffuse reflectance component in the living body and k values on 550 nm and 1,450 nm, the diffuse reflectance component on 1,450 nm is about 10⁻³ of the diffuse reflectance component on 550 nm. If specular reflectance is calculated form refractive index of water and refractive index of air using n values on 550 nm and 1,450 nm, specular reflectance on 1,450 nm and specular reflectance on 550 nm are respectively 0.0189 and 0.0206 and thus approximately equal to each other. The specular light on 1,450 nm is about 100 times the diffuse reflectance component. In this way, the specular light, namely, the surface reflection component is dominant in infrared light in the SWIR range, such as on 1,450 nm. The diffuse reflectance component causing the image contrast serving a spatial resolution to be decreased is substantially reduced, thereby increasing the spatial resolution.

In imaging through visible light, blue light hard to be absorbed, in particular, by water, is diffused and reflected, resulting in an image with the outline of the shape thereof blurred. On the other hand, through imaging through the wavelength region of infrared, a surface shape and wrinkles of the skin may be more easily detected as a feature value. Increasing feature value information may increase accuracy of the impersonation determination and personal authentication. Since the diffuse reflectance component is reduced more in a wavelength having a higher absorption coefficient by water, an increase in the spatial resolution is more pronounced in infrared light in a wavelength range equal to or higher than 1,200 nm where the absorption coefficient k by water is particularly higher. The increase in the spatial resolution may lead to an increase in the authentication accuracy of the human face.

### Wavelength Range of Infrared Light

The wavelength range of infrared light used to image the first infrared image, namely, the wavelength range including the first wavelength is described below. In the following discussion, specific numerical values about the first wavelength are described. A wavelength of interest is not necessarily strictly defined according to a unit of 1 nm and any wavelength falling within a range of 50 nm located on the wavelength of interest may be acceptable. This is because the wavelength characteristics of a light source and an imager do not necessarily exhibit a sharp response at a resolution as precise as a unit of several nm.

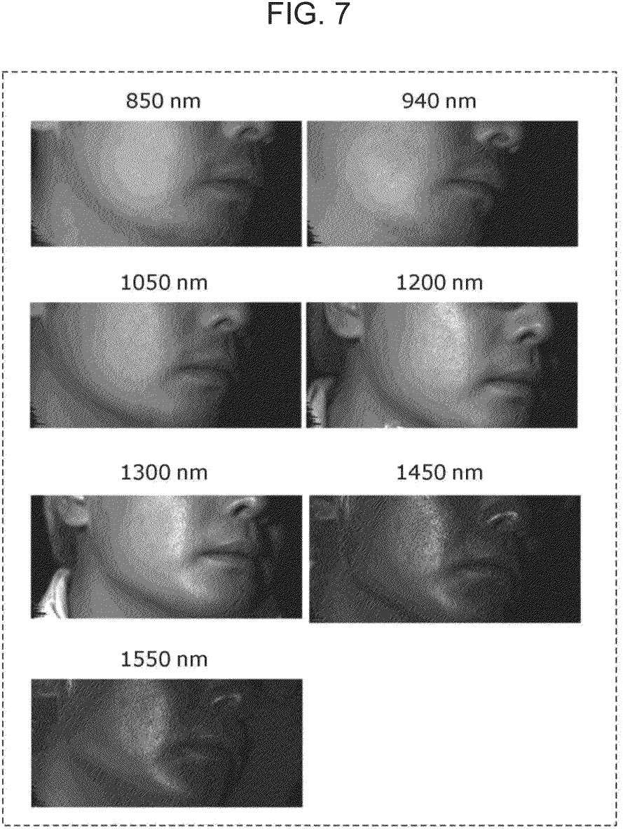

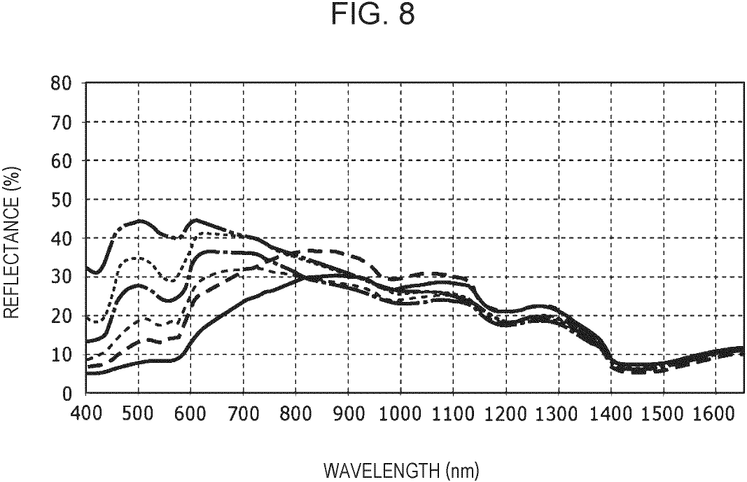

FIG. 7 illustrates images of the human face on 850 nm, 940 nm, 1,050 nm, 1200 nm, 1,300 nm, 1,450 nm, and 1,550 nm. FIG. 8 illustrates a wavelength dependency of reflectance of light on the color of skin. FIG. 8 illustrates data written on Holger Steiner, “Active Multispectral SWIR Imaging for Reliable Skin Detection and Face Verification,” Cuvillier Verlag, Jan. 10, 2017, pp. 13-14. Referring to FIG. 8, graphs different in type of lines from skin color to skin color are illustrated.

The first wavelength is 1,100 nm or shorter. In this way, an imaging device including a low-cost silicon sensor may be used to image the subject. Since a wavelength range from 850 nm to 940 nm has been recently widely used in ranging systems, such as time of flight (ToF) methods, a configuration including a light source may be implemented at a lower cost.

As illustrated in FIG. 7, wavelengths 850 nm, 940 nm, and 1,050 nm may allow subcutaneous blood vessels to be clearly observed. The comparison of the visible light image and the first infrared image may thus determine whether the subject is a living body, or an artificial object made of paper or silicone rubber.

The first wavelength may be, for example, 1,100 nm or longer. Referring to FIG. 8, there is no or little difference in light reflectance dependent on the skin color in the wavelength on 1,100 nm or longer. Since the light reflectance is less affected by a difference in the skin color and hair color, a stable biometric authentication system 1 globally accepted may result.

The first wavelength may be, for example, 1,200 nm or longer. Since the absorption of infrared light by the water component in the living body increases on the wavelength 1,200 nm or longer, the contrast of the first infrared image becomes clearer as illustrated in FIG. 7. The impersonation determination may be performed at a higher accuracy. The ratio of the surface reflection component to the diffuse reflectance component in the light reflected from the living body becomes higher and the spatial resolution of the first infrared image increases. The accuracy of the personal authentication using the first infrared image may be increased. The principle for this reason has been described with reference to FIGS. 4 through 6.

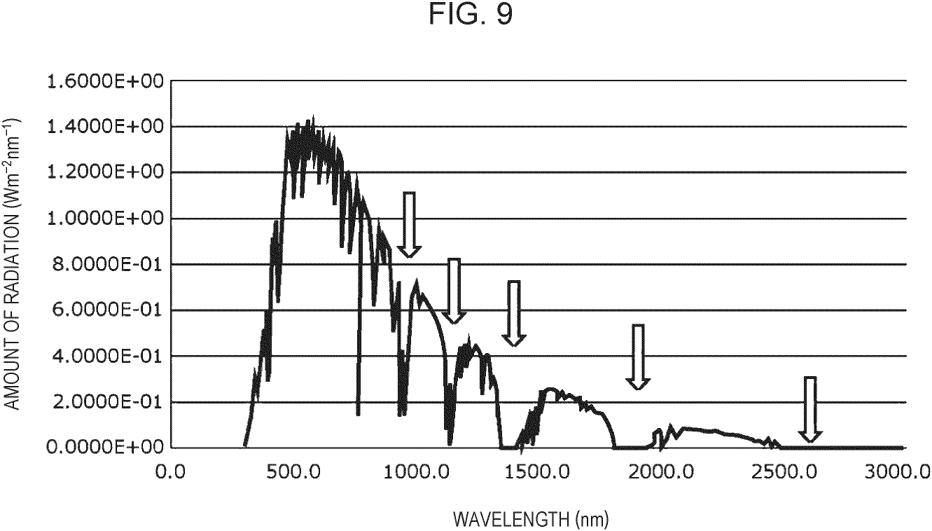

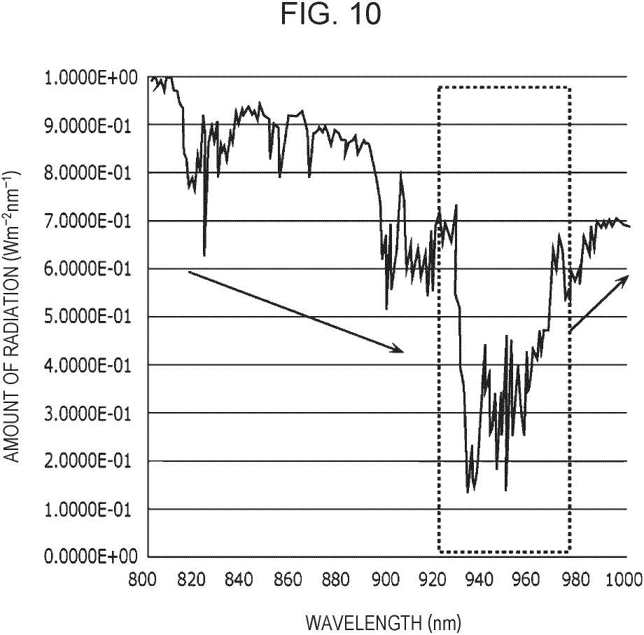

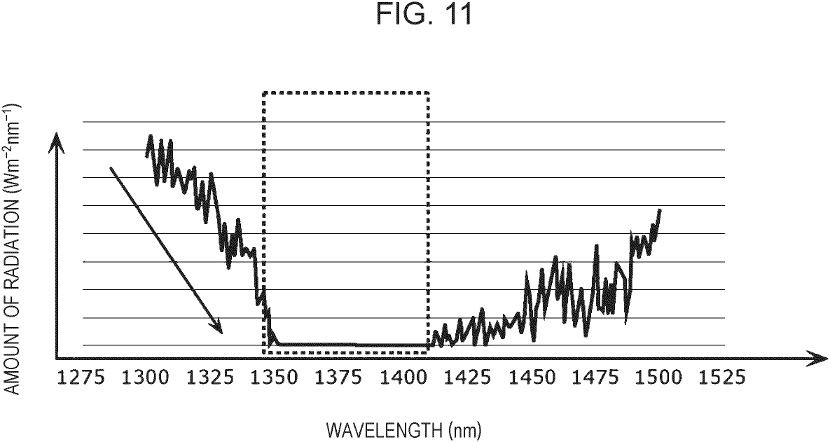

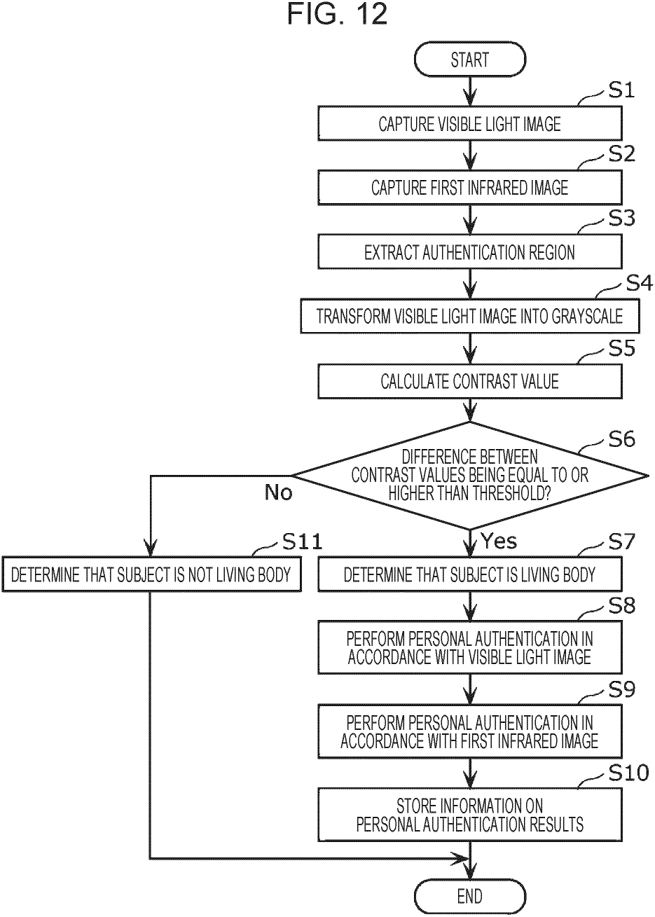

The first wavelength may be determined from the standpoint of the missing wavelength range of the sunlight. FIG. 9 illustrates a sunlight spectrum on the ground. FIG. 10 illustrates in enlargement a portion of the sunlight spectrum in FIG. 9. FIG. 11 illustrates in enlargement another portion of the sunlight spectrum in FIG. 9. Referring to FIG. 9, a portion of wavelength range on the ground has the missing part of the sunlight attributed to light absorption through the atmospheric layer and a water component in the air on the ground. When imaging is performed in a narrow-band wavelength using any active light illuminator, such as the first light illuminator 410, the use of the wavelength in the missing part may control imaging through the effect of ambient light that is not intended and is outside the irradiation light from the active light illuminator. Specifically, imaging having no or little effect of ambient light may be performed. Since the first infrared image obtained through imaging using light reflected in the narrow-band wavelength region including the first wavelength is used, the biometric authentication system 1 may thus increase the accuracy of the impersonation determination and the personal authentication.

In view of the missing wavelength of the sunlight, the first wavelength may be in the vicinity of 940 nm, specifically, is equal to or longer than 920 nm and equal to or shorter than 980 nm. Referring to FIGS. 9 and 10, the wavelength range in the vicinity of 940 nm has a weaker wavelength component of the sunlight on the ground. Since the effect of the sunlight is small in comparison with other wavelength, disturbance from the sunlight is less likely and a stable biometric authentication system 1 may thus be constructed. In the wavelength range equal to or longer than 920 nm and equal to or shorter than 980 nm, an amount of radiation on the ground is higher than in a wavelength range to be discussed below, but absorption of light in the atmosphere is smaller. The reduction in light in the active light illuminator, such as the first light illuminator 410, is also smaller. Since the first wavelength is equal to or shorter than 1,100 nm, a low-cost configuration may be implemented as described above.

In view of the missing wavelength of the sunlight, the first wavelength may be in the vicinity of 1,400 nm, specifically, is equal to or longer than 1,350 nm and equal to or shorter than 1,450 nm. Referring to FIGS. 9 and 11, the wavelength range equal to or longer than 1,350 nm and equal to or shorter than 1,450 nm of the sunlight, in particular, the wavelength range equal to or longer than 1,350 nm and equal to or shorter than 1,400 nm has a pronounced effect of the missing part of the sunlight in comparison with the wavelength in the vicinity of 940 nm and is less likely to be influenced by ambient light noise. As previously described, the wavelength in the vicinity of 1,400 nm increases the absorption by the water component of the living body, and provides a clearer contrast, thereby implementing the impersonation determination at a higher accuracy. Since the spatial resolution is increased, accuracy of the personal authentication is also increased. For example, with reference to FIG. 3, the color of the skin in the image imaged by imaging the infrared light of 1,450 nm appears darker because of the absorption by the water component. A determination as to whether the subject is a living body may be more easily performed by comparing the contrast values or luminance values of the visible light image and the first infrared image.

On the other hand, the absorption of the irradiation light from the active light illuminator, such as the first light illuminator 410 in the atmosphere is relatively higher in the wavelength in the vicinity of 1,400 nm. In view of this, the shortest wavelength in the light emission spectrum of the first light illuminator 410 is shifted to a short wavelength side shorter than 1,350 nm or the longest wavelength is shifted to a long wavelength side longer than 1,400 nm. Imaging may thus be performed with the ambient light noise reduced and the absorption of the irradiation light in the atmosphere restricted.

The missing wavelength of the sunlight in the vicinity of 940 nm or 1,400 nm may be used. Imaging in the narrow-band wavelength using a desired missing wavelength of the sunlight may be performed by setting the half width of a spectral sensitivity peak of the second imaging device 312 to be equal to or shorter than 200 nm or by setting the width at 10% of a maximum spectral sensitivity of the spectral sensitivity peak to be equal to or shorter than 200 nm.

The missing wavelength of the sunlight is cited as an example only. Referring to FIG. 9, the first wavelength may be a wavelength in each of the wavelength regions respectively including 850 nm, 1,900 nm, or 2,700 nm or a wavelength longer than each of these wavelengths.

### Process

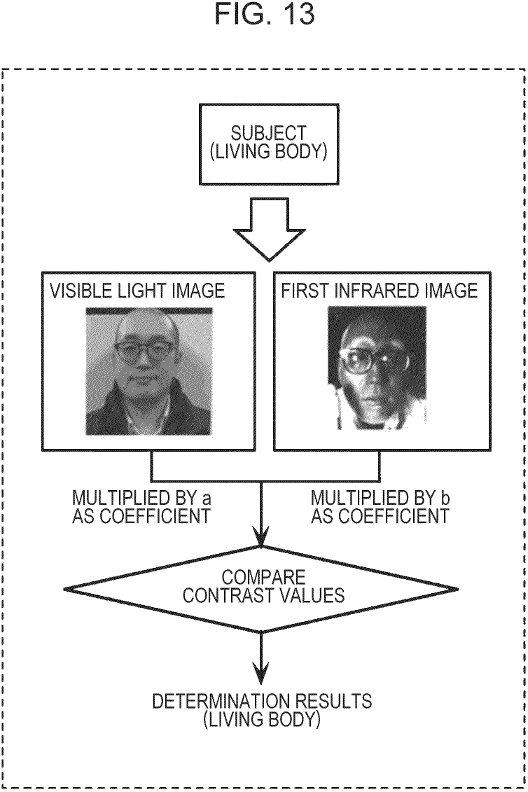

Process of the biometric authentication system 1 is described below. FIG. 12 is a flowchart illustrating a process example of the biometric authentication system 1 of the first embodiment. Specifically, the process example illustrated in FIG. 12 is performed by the processor 100 in the biometric authentication system 1.

The first image capturer 111 captures the visible light image (step S1). For example, the first imaging device 311 images the visible light image by picking up light reflected from the subject irradiated with visible light. The first image capturer 111 captures the visible light image picked up by the first imaging device 311.

The second image capturer 112 captures the first infrared image (step S2). For example, the first light illuminator 410 irradiates the subject with infrared light within a wavelength range including the first wavelength. The second imaging device 312 images the first infrared image by picking up light that is reflected from the subject irradiated with infrared light by the first light illuminator 410 and includes the wavelength region including the first wavelength. For example, the timing controller 500 outputs the first synchronization signal to the second imaging device 312 and the first light illuminator 410 and the second imaging device 312 images the first infrared image in synchronization with the irradiation of infrared light of the first light illuminator 410. The second image capturer 112 thus captures the first infrared image imaged by the second imaging device 312.

The second imaging device 312 may image multiple first infrared images. For example, the second imaging device 312 under the control of the timing controller 500 images two first infrared images when the first light illuminator 410 emits infrared light and when the first light illuminator 410 does not emit infrared light. The determiner 120 or the like determines a difference between the two first infrared images, leading to an image with the ambient light offset. The resulting image may be used in the impersonation determination and the personal authentication.

The determiner 120 extracts an authentication region having the photographed subject from each of the visible light image captured by the first image capturer 111 and the first infrared image captured by the second image capturer 112 (step S3). If the subject is a human face, the determiner 120 detects a face in each of the visible light image and the first infrared image and extracts as the authentication region a region where the detected face is depicted. The face detection method may be any of related-art techniques that detect face in accordance with features of image.

The region to be extracted may not necessarily be an entire region where the entire face is depicted. A region depicting a portion typically representing the face, for example, a region depicting at least a portion selected from the group consisting of eyebrows, eyes, cheeks, and forehead, may be extracted. Processing may proceed to step S4 with the operation in step S3 extracting the authentication region skipped.

The determiner 120 transforms the visible light image with the authentication region extracted in step S3 to grayscale (step S4). The determiner 120 may also transform the first infrared image with the authentication region extracted to grayscale. In such a case, the visible light image with the authentication region extracted and the first infrared image with the authentication region extracted are grayscale-transformed on the same level quantization (for example, 16-level quantization). This causes the two image to match in luminance scale, reducing workload in subsequent process. The visible light image and the first infrared image having undergone the operations in steps S1 through S4 are respectively referred to as a determination visible light image and a determination first infrared image.

The operation in step S4 may be skipped when the visible light image is a grayscale image and the visible light image and the first infrared image may be respectively used as the determination visible light image and the determination first infrared image.

The determiner 120 calculates contrast values from the determination visible light image and the determination first infrared image (step S5). Specifically, the determiner 120 multiplies each luminance value (in other words, each pixel value) of the determination visible light image by a coefficient a, and each luminance value of the determination first infrared image by a coefficient b. The coefficient a and the coefficient b are set in response to an imaging environment and the first wavelength such that the determination visible light image matches the determination first infrared image in brightness. For example, the coefficient a may be set to be smaller than the coefficient b. The determiner 120 calculates the contrast values of the images using the luminance values of the determination visible light image and the determination first infrared image that are respectively multiplied by the coefficients. Let Pmax represent a maximum luminance value of the image and Pmin represent a minimum luminance value, and the contrast value is the contrast value = (Pmax - Pmin) / (Pmax + Pmin).

The determiner 120 determines whether a difference between the contrast value of the determination visible light image and the contrast value of the determination first infrared image calculated in step S5 is equal to or higher than a threshold (step S6). The threshold in step S6 may be set in view of the imaging environment, the first wavelength, and the purpose of the impersonation determination.

If the difference between the contrast value of the determination visible light image and the contrast value of the determination first infrared image is equal to or higher than the threshold (yes path in step S6), the determiner 120 determines that the subject is a living body, and then outputs determination results to the first authenticator 131, the second authenticator 132, and the outside (step S7). If the subject is a living body, the contrast value of the determination first infrared image increases under the influence of the absorption by the water component. For this reason, if the contrast value of the determination first infrared image is larger than the contrast value of the determination visible light image by the threshold, the determiner 120 determines that the subject is a living body, in other words, the subject is not impersonated.

If the difference between the contrast value of the determination visible light image and the contrast value of the determination first infrared image is larger than the threshold (no path in step S6), the determiner 120 determine that the subject is not a living body, and outputs the determination results to the first authenticator 131, the second authenticator 132, and the outside (step S11). If the subject is an artificial object, the contrast value of the determination first infrared image is not so high as when the subject is a living body. If the contrast value of the determination first infrared image is not larger than the contrast value of the determination visible light image by the threshold, the determiner 120 determines that the subject is not a living body, namely, determines that the subject is impersonated.

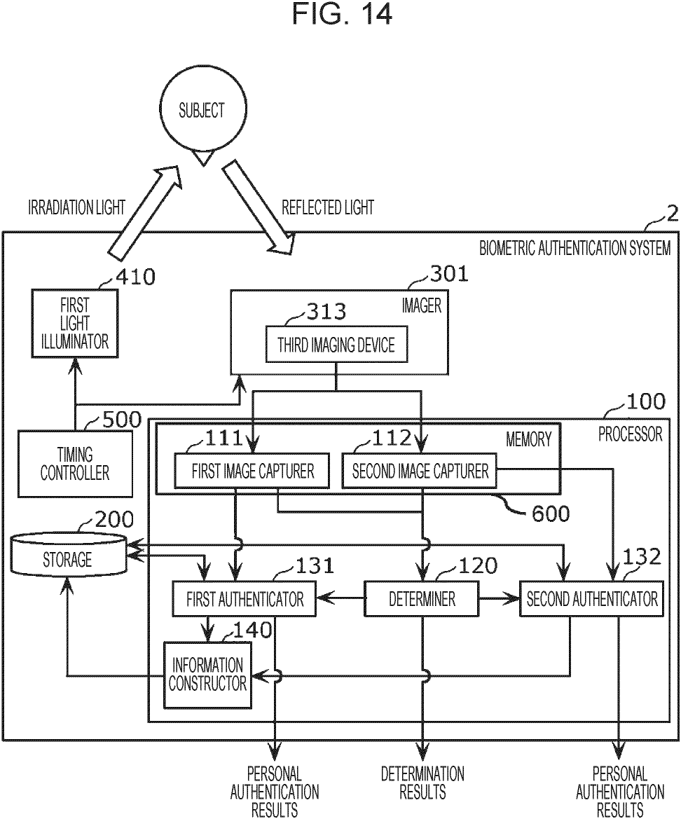

FIG. 13 illustrates how the biometric authentication system 1 performs the impersonation determination when the subject is not impersonated. Referring to FIG. 13, if the subject is a living body, the biometric authentication system 1 acquires the visible light image and the first infrared image that are very different in terms of contrast value. As described above, the biometric authentication system 1 performs a determination as to whether the subject is impersonated, by multiplying the luminance value of the visible light image by the coefficient a, by multiplying the luminance value of the first infrared image by the coefficient b, and then by comparing the contrast values. With reference to FIG. 13, the subject is a living body, the difference between the contrast values is larger than the threshold, and the determination results indicating that the subject is not impersonated are output. In this way, the biometric authentication system 1 performs the impersonation determination at a higher accuracy using the contrast values that are easily calculated.

Referring back to FIG. 12, the first authenticator 131 acquires determination results indicating that the determiner 120 determines in step S7 that the subject is a living body, performs the personal authentication on the subject in accordance with the visible light image, and outputs results of the personal authentication (step S8). The first authenticator 131 performs the personal authentication as to whether to authenticate, by checking the visible light image against the image of the subject registered in a personal authentication database on the storage 200. The method of the personal authentication may be a related-art method of extracting and sorting feature values through machine learning. If the subject is a human face, the personal authentication is performed by extracting the feature values of the face, such as the eyes, the nose, and the mouth and by checking the feature values according to locations and sizes of the feature values. When the first authenticator 131 performs the personal authentication on the subject in accordance with the visible light image, a sufficient visible light image database is available. The biometric authentication system 1 may thus perform the personal authentication at a higher accuracy.

The second authenticator 132 acquires the determination results indicating that the determiner 120 determines in step S7 that the subject is a living body, performs the personal authentication on the subject in accordance with the first infrared image, and outputs the results of the personal authentication to the outside (step S9). The personal authentication method performed by the second authenticator 132 is the same authentication method as the first authenticator 131. As described above, since the ratio of the surface reflection component to the diffuse reflectance component of the light reflected from the living body irradiated with light is higher in infrared light than in visible light, the first infrared image has a higher spatial resolution than the visible light image. The biometric authentication performed in accordance with the first infrared image at a higher spatial resolution may provide a higher accuracy in the personal authentication.

The information constructor 140 stores information on the results of the biometric authentication performed by the first authenticator 131 and information on the results of the biometric authentication performed by the second authenticator 132 in an associated form on the storage 200 (step S10). For example, the information constructor 140 registers the visible light image and the first infrared image authenticated through the personal authentication in an associated form in the personal authentication database on the storage 200. The information stored by the information constructor 140 is related to results obtained through highly reliable personal authentication indicating that the subject is not impersonated. In this way, the database storing infrared images having a relatively higher spatial resolution than visible light images but a relatively smaller amount of information than visible light images may be expanded. Machine learning using these pieces of information may construct a biometric authentication system 1 that performs the personal authentication at a higher accuracy. After step S10, the processor 100 in the biometric authentication system 1 ends the process.

On the other hand, when the determiner 120 determines in step S11 that the subject is not a living body, the processor 100 in the biometric authentication system 1 ends the process. Specifically, when the determiner 120 determines that the subject is not a living body, the first authenticator 131 and the second authenticator 132 do not perform the personal authentication on the subject. If the subject is not impersonated, the personal authentication is performed while if the subject is impersonated, the personal authentication is not performed. This may lead to a reduction in the workload of the processor 100.

The first authenticator 131 and the second authenticator 132 may perform the personal authentication regardless of whether the determination results of the determiner 120. In such a case, the personal authentication may be performed without waiting for the determination results from the determiner 120. This allows both the impersonation determination and the personal authentication to be performed in parallel, thereby increasing a processing speed of the processor 100.

As described above, the biometric authentication system 1 determines in accordance with the visible light image and the first infrared image whether the subject is a living body. With only the two types of images, the impersonation determination may be performed. The biometric authentication system 1 may thus be down-sized. Regardless of whether the subject impersonated is a planar shape or a three-dimensional shape, the impersonation determination may be easily performed in accordance with a difference in the contrasts or other factors between the visible light image and the first infrared image. The impersonation determination may thus be performed at a higher accuracy. A down-sized biometric authentication system 1 having an higher authentication accuracy may thus result.

### Modification

A biometric authentication system as a modification of the first embodiment is described below. The following discussion focuses on a difference between the first embodiment and the modification thereof and the common parts therebetween are briefly described or not described at all.

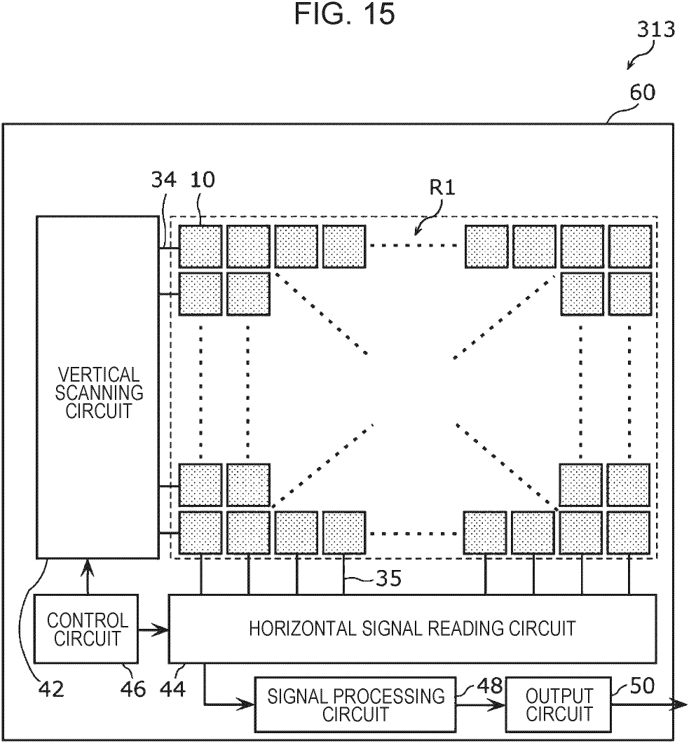

FIG. 14 is a block diagram illustrating a functional configuration of a biometric authentication system 2 according to the modification of the first embodiment.

Referring to FIG. 14, the biometric authentication system 2 of the modification is different from the biometric authentication system 1 of the first embodiment in that the biometric authentication system 2 includes an imager 301 in place of the imager 300.

The imager 301 includes a third imaging device 313 that images the visible light image and the first infrared image. The third imaging device 313 may be implemented by an imager having a photoelectric conversion layer having a spectral sensitivity to visible light and infrared light. The third imaging device 313 may be a camera, such as an indium gallium arsenide (InGaAs) camera, having a spectral sensitivity to both visible light and infrared light. Since the imager 301 including a single third imaging device 313 is enabled to image both the visible light image and the first infrared image, the biometric authentication system 2 may be down-sized. Since the third imaging device 313 images in a coaxial manner both the visible light image and the first infrared image, the effect of parallax may be controlled by the visible light image and the first infrared image, leading to a biometric authentication system 2 at higher accuracy authentication.

In the biometric authentication system 2, the first image capturer 111 captures the visible light image from the third imaging device 313 and the second image capturer 112 captures the first infrared image from the third imaging device 313.

The timing controller 500 in the biometric authentication system 2 controls an imaging timing of the imager 301 and an irradiation timing of the first light illuminator 410. The timing controller 500 outputs the first synchronization signal to the third imaging device 313 and the first light illuminator 410. The third imaging device 313 images the first infrared image at a timing responsive to the first synchronization signal. The first light illuminator 410 irradiates the subject with infrared light at the timing responsive to the first synchronization signal. In this way, the timing controller 500 causes the third imaging device 313 to image the first infrared image while the first light illuminator 410 irradiates the subject with infrared light.

The biometric authentication system 2 operates in the same way as the biometric authentication system 1 except that the first image capturer 111 and the second image capturer 112 respectively capture the visible light image and the first infrared image from the third imaging device 313 in the biometric authentication system 2.

A specific configuration of the third imaging device 313 is described below.

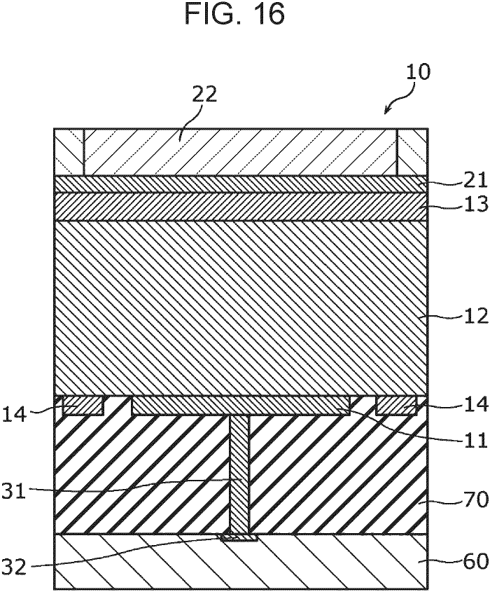

FIG. 15 illustrates a configuration example of the third imaging device 313 according to the modification of the first embodiment. The third imaging device 313 in FIG. 15 includes multiple pixels 10 and peripheral circuits formed on a semiconductor substrate 60. According to the modification of the first embodiment, the third imaging device 313 is a lamination-type imaging device in which a photoelectric conversion layer and electrodes are laminated.

Each pixel 10 includes a first photoelectric conversion layer 12 that is above the semiconductor substrate 60 as described below. The first photoelectric conversion layer 12 serves as a photoelectric converter that generates pairs of holes and electrons in response to incident light. Referring to FIG. 15, the pixels 10 are spaced apart from each other for convenience of explanation. It is contemplated that the pixels 10 are continuously arranged with no spacing therebetween on the semiconductor substrate 60. Each pixel 10 may include a photodiode formed as a photoelectric converter in the semiconductor substrate 60.

Referring to FIG. 15, the pixels 10 are arranged in a matrix of m rows and n columns. Each of m and n represents an integer equal to 1 or higher. The pixels 10 are two-dimensionally arranged on the semiconductor substrate 60, forming an imaging region R1. The imaging region R1 includes the pixels 10 that include optical filters 22 different from each other in transmission wavelength range and respectively used for infrared light within a wavelength range including the first wavelength, blue light, green light, and red light. In this way, image signals respectively responding to the infrared light within the wavelength range including the first wavelength, blue light, green light, and red light are separately read. The third imaging device 313 generates the visible light image and the first infrared image using these image signals.

The number and layout of the pixels 10 are illustrated but the disclosure is not limited to the arrangement illustrated in FIG. 15. The center of each pixel 10 is centered on a lattice point of each square lattice. Alternatively, the pixels 10 may be arranged such that the center of each pixel 10 may be at the lattice point of a triangular lattice or a hexagonal lattice.

The peripheral circuits include, for example, a vertical scanning circuit 42, a horizontal signal reading circuit 44, a control circuit 46, a signal processing circuit 48, and an output circuit 50. The peripheral circuits may further include a voltage supply circuit that supplies power to the pixels 10.

The vertical scanning circuit 42 may also be referred to as a row scanning circuit and is connected to each of address signal lines 34 respectively arranged for rows of the pixels 10. The signal line arranged for each row of the pixels 10 is not limited to the address signal line 34. Multiple types of signal lines may be connected to each row of the pixels 10. The vertical scanning circuit 42 selects the pixels 10 by row by applying a predetermined voltage to the address signal line 34, reads a signal voltage and performs a reset operation.

The horizontal signal reading circuit 44 is also referred to as a column scanning circuit and is connected to each of vertical scanning lines 35 respectively arranged for columns of the pixels 10. An output signal from the pixels 10 selected by row by the vertical scanning circuit 42 is read onto the horizontal signal reading circuit 44 via the vertical scanning line 35. The horizontal signal reading circuit 44 performs on the output signal from the pixel 10a noise suppression and signal processing operation, such as correlated double sampling, and analog-to-digital (AD) conversion operation.

The control circuit 46 receives instruction data and clock from the outside and controls the whole third imaging device 313. The control circuit 46 including a timing generator supplies a drive signal to the vertical scanning circuit 42, the horizontal signal reading circuit 44, and the voltage supply circuit. The control circuit 46 may be implemented by a microcontroller including one or more processors storing a program. The function of the control circuit 46 may be implemented by a combination of a general-purpose processing circuit and a software component or by a hardware component that is specialized in the process of the control circuit 46.

The signal processing circuit 48 performs a variety of operations on an image signal acquired from the pixel 10. In the context of the specification, the “image signal” is an output signal used to form an image among signals read via the vertical scanning line 35. The signal processing circuit 48 generates an image in accordance with the image signal read by, for example, the horizontal signal reading circuit 44. Specifically, the signal processing circuit 48 generates the visible light image in accordance with the image signals from the pixels 10 that photoelectrically converts visible light, and generates the first infrared image in accordance with the image signals from the pixels 10 that photoelectrically converts infrared light. The outputs from the signal processing circuit 48 are read to the outside of the third imaging device 313 via the output circuit 50. The signal processing circuit 48 may be implemented by a microcontroller including one or more processors storing a program. The function of the signal processing circuit 48 may be implemented by a combination of a general-purpose processing circuit and a software component or by a hardware component that is specialized in the process of the signal processing circuit 48.

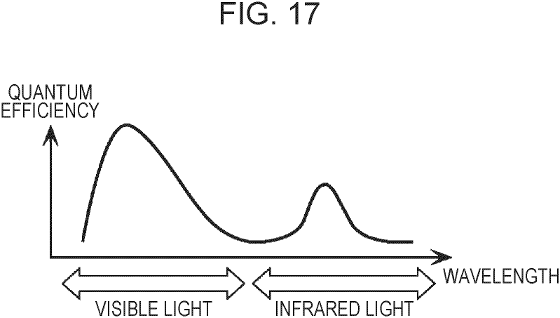

The cross-sectional structure of the pixel 10 in the third imaging device 313 is described below. FIG. 16 is a schematic cross-sectional view illustrating a cross-sectional structure of the pixel 10 of the third imaging device 313 according to the modification of the first embodiment. The pixels 10 are identical to each other in structure except that transmission wavelength of each optical filter 22 is different. Some of the pixels 10 may be different from the rest of the pixels 10 not only in the optical filter 22 but also in another portion.

Referring to FIG. 16, the pixel 10 includes the semiconductor substrate 60, a pixel electrode 11 disposed above the semiconductor substrate 60 and respectively electrically connected to the semiconductor substrate 60, a counter electrode 13 above the pixel electrode 11, a first photoelectric conversion layer 12 interposed between the pixel electrode 11 and the counter electrode 13, an optical filter 22 disposed above the counter electrode 13, and a charge accumulation node 32 electrically connected to the pixel electrode 11 and accumulating signal charges generated by the first photoelectric conversion layer 12. The pixel 10 may further include a sealing layer 21 disposed between the counter electrode 13 and the optical filter 22, and auxiliary electrodes 14 facing the counter electrode 13 with the first photoelectric conversion layer 12 interposed therebetween. Light is incident on the pixel 10 from above the semiconductor substrate 60.

The semiconductor substrate 60 is a p-type silicon substrate. The semiconductor substrate 60 is not limited to a substrate that is entirely semiconductor. A signal detector circuit (not illustrated in FIG. 16) including transistors detecting signal charges generated by the first photoelectric conversion layer 12 is disposed on the semiconductor substrate 60. The charge accumulation node 32 is a portion of the signal detector circuit and a signal voltage responsive to an amount of signal charges accumulated on the charge accumulation node 32 is read.

An interlayer insulation layer 70 is disposed on the semiconductor substrate 60. The interlayer insulation layer 70 is manufactured of an insulating material, such as silicon dioxide. The interlayer insulation layer 70 may include a signal line (not illustrated), such as the vertical scanning line 35, or a power supply line (not illustrated). The interlayer insulation layer 70 includes a plug 31. The plug 31 is manufactured of an electrically conductive material.

The pixel electrode 11 collects signal charges generated by the first photoelectric conversion layer 12. Each pixel 10 includes at least one pixel electrode 11. The pixel electrode 11 is electrically connected to the charge accumulation node 32 via the plug 31. The signal charges collected by the pixel electrode 11 are accumulated on the charge accumulation node 32. The pixel electrode 11 is manufactured of an electrically conductive material. The electrically conductive material may be a metal, such as aluminum or copper, metal nitride, or polysilicon to which conductivity is imparted through impurity doping.

The first photoelectric conversion layer 12 absorbs visible light and infrared light within a wavelength range including the first wavelength and generates photocharges. Specifically, the first photoelectric conversion layer 12 has a spectral sensitivity to the first wavelength and a wavelength range of visible light. Specifically, the first photoelectric conversion layer 12 receives incident light and generates hole-electron pairs. Signal charges are either holes or electrons. The signal charges are collected by the pixel electrode 11. Charges in polarity opposite to the signal charges are collected by the counter electrode 13. In the context of the specification, having a spectral sensitivity to a given wavelength signifies that external quantum efficiency of the wavelength is equal to or higher than 1%.

Since the first photoelectric conversion layer 12 has a spectral sensitivity to the first wavelength and the wavelength range of visible light, the third imaging device 313 may image the visible light image and the first infrared image. The first photoelectric conversion layer 12 has a spectral sensitivity peak on the first wavelength.

The first photoelectric conversion layer 12 contains a donor material that absorbs light within the wavelength range including the first wavelength and light within the wavelength range of visible light, and generates hole-electron pairs. The donor material contained in the first photoelectric conversion layer 12 is an inorganic semiconductor material or an organic semiconductor material. Specifically, the donor material contained in the first photoelectric conversion layer 12 is semiconductor quantum dots, semiconductor carbon nanotubes, and/or an organic semiconductor material. The first photoelectric conversion layer 12 may contain one or more types of donor materials. Multiple types of donor materials, if contained in the first photoelectric conversion layer 12, may be a mixture of a donor material absorbing infrared light within the wavelength range including the first wavelength and a donor material absorbing visible light.

The first photoelectric conversion layer 12 contains, for example, a donor material and semiconductor quantum dots. The semiconductor quantum dots have a three-dimensional quantum confinement effect. The semiconductor quantum dots are nanocrystals, each having a diameter of from 2 nm to 10 nm and including dozens of atoms. The material of the semiconductor quantum dots is group IV semiconductor, such as Si or Ge, group IV-VI semiconductor, such as PbS, PbSe, or PbTe, group III-V semiconductor, such as InAs or InSb, or ternary mixed crystals, such as HgCdTe or PbSnTe.

The semiconductor quantum dots used in the first photoelectric conversion layer 12 has the property of absorbing light within the wavelength range of infrared light and the wavelength range of visible light. The absorption peak wavelength of the semiconductor quantum dots is attributed to an energy gap of the semiconductor quantum dots and is controllable by a material and a particle size of the semiconductor quantum dots. The use of the semiconductor quantum dots may easily adjust the wavelength to which the first photoelectric conversion layer 12 has a spectral sensitivity. The absorption peak of the semiconductor quantum dots within the wavelength range of infrared light is a sharp peak having a half width of 200 nm or lower and thus the use of the semiconductor quantum dots enables imaging to be performed in a narrow-band wavelength within the wavelength range of infrared light. Since the material of the semiconductor carbon nanotubes has the quantum confinement effect, the semiconductor carbon nanotubes have a sharp absorption peak in the wavelength range of infrared light as the semiconductor quantum dots do. The material having the quantum confinement effect enables imaging to be performed in the narrow-band wavelength within the wavelength range of infrared light.

The materials of the semiconductor quantum dots exhibiting an absorption peak within the wavelength range of infrared light may include, for example, PbS, PbSe, PbTe, InAs, InSb, Ag₂S, Ag₂Se, Ag₂Te, CuS, CuInS₂, CuInSe₂, AgInS₂, AgInSe₂, AgInTe₂, ZnSnAs₂, ZnSnSb₂, CdGeAs₂, CdSnAs₂, HgCdTe, and InGaAs. The semiconductor quantum dots used in the first photoelectric conversion layer 12 have, for example, an absorption peak on the first wavelength.

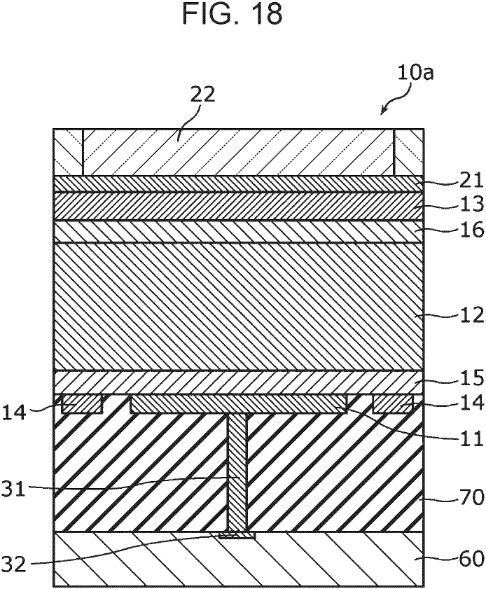

FIG. 17 schematically illustrates a spectral sensitivity curve of the pixel 10. Specifically, FIG. 17 illustrates a relationship between the external quantum efficiency of the first photoelectric conversion layer 12 containing the semiconductor quantum dots and the wavelength of light. Referring to FIG. 17, the first photoelectric conversion layer 12 has a spectral sensitivity to the wavelength range of visible light and the wavelength range of infrared light in response to the absorption wavelength of the semiconductor quantum dots. Since the first photoelectric conversion layer 12 containing the semiconductor quantum dots has the spectral sensitivity to the wavelength range of visible light and the wavelength range of infrared light, the third imaging device 313 simply including the first photoelectric conversion layer 12 as a photoelectric conversion layer is enabled to image the visible light image and the first infrared image.

The first photoelectric conversion layer 12 may include multiple types of semiconductor quantum dots different in terms of particle size and/or multiple types of semiconductor quantum dots different in terms of material.

The first photoelectric conversion layer 12 may further contain an acceptor material that accepts electrons from the donor material. Since electrons from hole-electron pairs generated in the donor material move to the acceptor material in this way, recombination of holes and electrons is controlled. The external quantum efficiency of the first photoelectric conversion layer 12 may be improved. The acceptor material may be C60 (fullerene), phenyl C₆₁ butyric acid methyl ester (PCBM), C60 derivatives such as indene C₆₀ bis adduct (ICBA), or oxide semiconductor, such as TiO₂, ZnO, or SnO₂.

The counter electrode 13 is a transparent electrode manufactured of a transparent conducting material. The counter electrode 13 is disposed on a side where light is incident on the first photoelectric conversion layer 12. The light transmitted through the counter electrode 13 is thus incident on the first photoelectric conversion layer 12. In the context of the specification, the word “transparent” signifies that at least part of light in the wavelength range to be detected is transmitted and does not necessarily signify that the whole wavelength range of visible light and infrared light is transmitted.

The counter electrode 13 is manufactured of a transparent conducting oxide (TCO), such as ITO, IZO, AZO, FTO, SnO₂, TiO₂, or ZnO. A voltage supply circuit supplies a voltage to the counter electrode 13. A voltage difference between the counter electrode 13 and the pixel electrode 11 is set and maintained to a desired value by adjusting the voltage that the voltage supply circuit supplies to the counter electrode 13.

The counter electrode 13 is formed across multiple pixels 10. This enables a control voltage of a desired magnitude from the voltage supply circuit to be supplied to the multiple pixels 10 at a time. If the control voltage of the desired magnitude from the voltage supply circuit is applied, the counter electrodes 13 may be separately arranged respectively for the pixels 10.

The controlling of the potential of the counter electrode 13 with respect to the potential of the pixel electrode 11 causes the pixel electrode 11 to collect, as signal charges, either holes or electrons of the pairs generated within the first photoelectric conversion layer 12 through photoelectric conversion. If the signal charges are holes, setting the potential of the counter electrode 13 to be higher than the potential of the pixel electrode 11 may cause the pixel electrode 11 to selectively collect holes. In the following discussion, holes are used as the signal charges. Alternatively, electrons may be used as the signal charges and in such a case, the potential of the counter electrode 13 is set to be lower than the potential of the pixel electrode 11.

The auxiliary electrode 14 is electrically connected to an external circuit not illustrated in FIG. 16 and collects a subset of signal charges generated by the first photoelectric conversion layer 12. For example, collecting signal charges generated in the first photoelectric conversion layer 12 between adjacent pixels 10 may control color mixture. This may lead to an improvement in the image quality of the visible light image and the first infrared image imaged by the third imaging device 313, thereby increasing the authentication accuracy of the biometric authentication system 2. The auxiliary electrode 14 may be manufactured using one of the conductive materials described with reference to the pixel electrode 11.

The optical filter 22 is disposed on each of the pixels 10. For example, the optical filter 22 having a transmission wavelength of a pixel 10 is arranged on that pixel 10. The transmission wavelength ranges of the optical filters 22 in the blue-light, green-light, and red-light pixels 10 used to generate the visible light image are the wavelength ranges respectively for corresponding light colors. The transmission wavelength range of the optical filters 22 in the pixels 10 used to generate the first infrared image is the wavelength range including the first wavelength of infrared light.

The optical filter 22 may be a long-pass filter that blocks light shorter than a specific wavelength and allows light longer than the specific wavelength to transmit therethrough. The optical filter 22 may also be a band-pass filter that allows light within a specific wavelength range to transmit therethrough and blocks light shorter than the wavelength range and light longer than the wavelength range. The optical filter 22 may be an absorbing filter, such as colored glass, or a reflective filter that is formed by laminating dielectric layers.

The third imaging device 313 may be manufactured using a typical semiconductor manufacturing process. In particular, when the semiconductor substrate 60 is a silicon substrate, a variety of silicon semiconductor processes may be used.

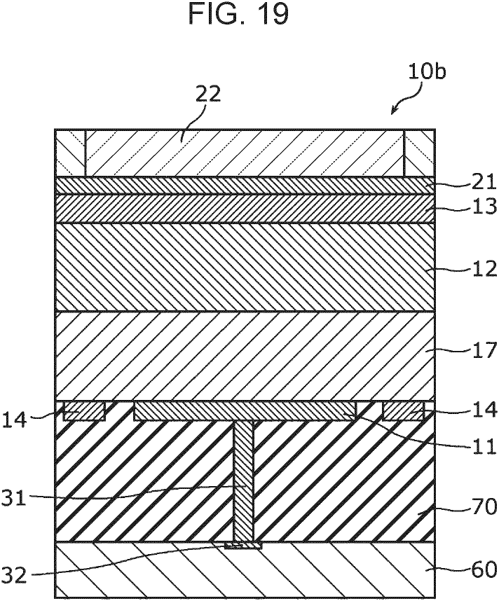

A pixel structure of the third imaging device 313 is not limited to the pixel 10 described above. Any pixel structure of the third imaging device 313 may be acceptable as long as the pixel structure is enabled to image the visible light image and the first infrared image. FIG. 18 is a schematic cross-sectional view illustrating a cross-sectional structure of another pixel 10a of the third imaging device 313 according to the modification of the first embodiment. The third imaging device 313 may include multiple pixels 10a in place of the pixels 10.

Referring to FIG. 18, the pixel 10a includes, besides the structure of the pixel 10, a hole transport layer 15 and a hole blocking layer 16.

The hole transport layer 15 is interposed between the pixel electrode 11 and the first photoelectric conversion layer 12. The hole transport layer 15 has a function of transporting holes as signal charges generated in the first photoelectric conversion layer 12 to the pixel electrode 11. The hole transport layer 15 may restrict the injection of electrons from the pixel electrode 11 to the first photoelectric conversion layer 12.

The hole blocking layer 16 is interposed between the counter electrode 13 and the first photoelectric conversion layer 12. The hole blocking layer 16 has a function of restricting the injection of holes from the counter electrode 13 to the first photoelectric conversion layer 12. The hole blocking layer 16 transports to the counter electrode 13 electrons in reverse polarity of the signal charges generated in the first photoelectric conversion layer 12.

The material of each of the hole transport layer 15 and the hole blocking layer 16 may be selected from related-art materials in view of a bonding strength with an adjacent layer, a difference in ionization potential, and an electron affinity difference, and the like.

Since the pixel 10a including the hole transport layer 15 and the hole blocking layer 16 is able to restrict the generation of dark currents, the image quality of the visible light image and the first infrared image imaged by the third imaging device 313 may be improved. The authentication accuracy of the biometric authentication system 2 may thus be increased.

If electrons are used as the signal charges, an electron transport layer and an electron blocking layer are respectively employed in place of the hole transport layer 15 and the hole blocking layer 16.

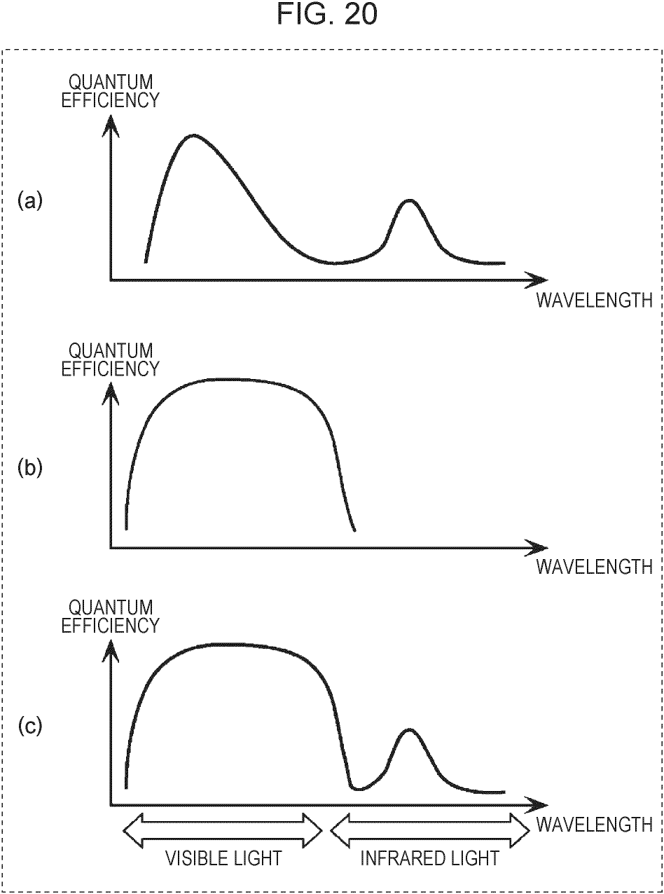

The third imaging device 313 may have a pixel structure including multiple photoelectric conversion layers. FIG. 19 is a schematic cross-sectional view illustrating a cross-sectional structure of another pixel 10b of the third imaging device 313 according to the modification of the first embodiment. The third imaging device 313 may include multiple pixels 10b in place of the pixels 10.

Referring to FIG. 19, the pixel 10b include, besides the structure of the pixel 10, a second photoelectric conversion layer 17.

The second photoelectric conversion layer 17 is interposed between the first photoelectric conversion layer 12 and the pixel electrode 11. The second photoelectric conversion layer 17 absorbs visible light and generates photocharges. The second photoelectric conversion layer 17 has a spectral sensitivity over the whole wavelength range of visible light. In the context of the specification, the whole wavelength range may be substantially the whole wavelength range of visible light. Specifically, wavelengths not used to image the visible light image, for example, a wavelength shorter than the wavelength used to output a luminance value of blue color and a wavelength longer than the wavelength used to output a luminance value of red color, may not be included in the whole wavelength range.

The second photoelectric conversion layer 17 contains a donor material that generates hole-electron pairs by absorbing the whole wavelength range of visible light. The donor material contained in the second photoelectric conversion layer 17 is a p-type semiconductor having a higher absorption coefficient in the wavelength range of visible light. For example, 2-{[7-(5-N, N-Ditolylaminothiophen-2-yl)-2, 1, 3-benzothiadiazol-4-yl]methylene}malononitrile (DTDCTB) has an absorption peak on or close to a wavelength of 700 nm, copper phthalocyanine and subphthalocyanine have respectively absorption peaks on or close to a wavelength of 620 nm and a wavelength of 580 nm, rubrene has an absorption peak on or close to a wavelength of 530 nm, α-sexithiophene has an absorption peak on or close to a wavelength of 440 nm. The absorption peak of each of these organic p-type semiconductor materials falls within the wavelength range of visible light and these p-type semiconductor materials may be used as the donor material of the second photoelectric conversion layer 17. If an organic material, such as one of these organic p-type semiconductor materials, is used, the location of the first photoelectric conversion layer 12 disposed closer to the light-incident side than the second photoelectric conversion layer 17 causes the first photoelectric conversion layer 12 to absorb part of the visible light. This may control the degradation of the organic material and durability of the second photoelectric conversion layer 17 may be increased.

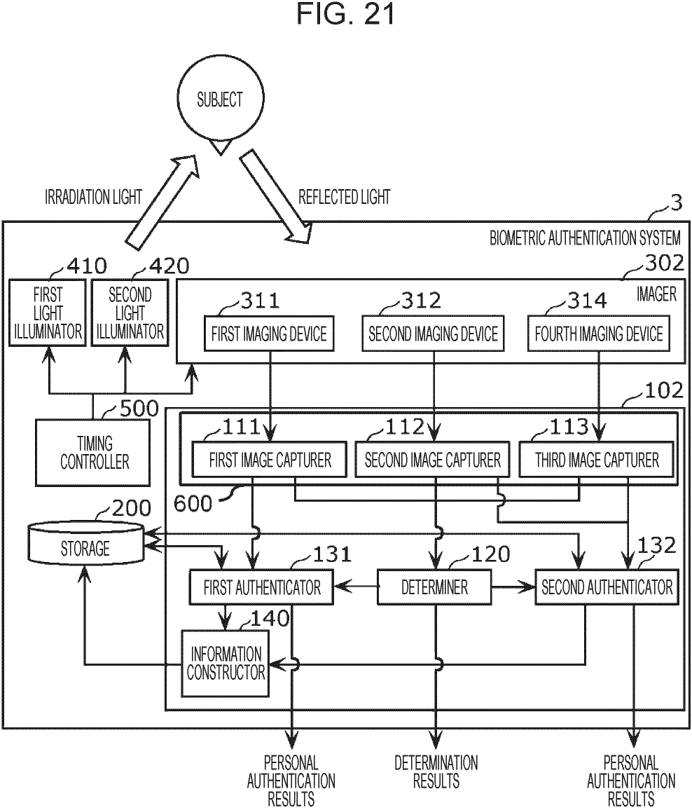

FIG. 20 schematically illustrates an example of spectral sensitivity curves of the pixel 10b according to the modification of the first embodiment. Part (a) of FIG. 20 illustrates a relationship between the external quantum efficiency of the first photoelectric conversion layer 12 and the wavelength of light. Part (b) of FIG. 20 illustrates a relationship between the external quantum efficiency of the second photoelectric conversion layer 17 and the wavelength of light. Part (c) of FIG. 20 illustrates a relationship between the external quantum efficiency of all the pixels 10b and the wavelength of light when the sensitivity of the first photoelectric conversion layer 12 and the sensitivity of the second photoelectric conversion layer 17 are combined.

The first photoelectric conversion layer 12 has a spectral sensitivity to the wavelength range of visible light and infrared light as illustrated in part (a) of FIG. 20 and the second photoelectric conversion layer 17 has, as illustrated in part (b) of FIG. 20, a spectral sensitivity to the wavelength range of visible light wider than the wavelength range of visible light to which the first photoelectric conversion layer 12 has a spectral sensitivity. Referring to part (c) of FIG. 20, all the pixels 10b has a spectral sensitivity to the whole wavelength range of infrared light and the whole wavelength range of visible light. The pixel 10b with the first photoelectric conversion layer 12 and the second photoelectric conversion layer 17 may provide an increase in the spectral sensitivity in a wider wavelength range and an improvement in the image quality of the visible light image and the first infrared image. In comparison with the case where the material of the first photoelectric conversion layer 12 and the material of the second photoelectric conversion layer 17 are included in a single photoelectric conversion layer, a decrease caused by interference between materials and color mixing between adjacent pixels 10b may be controlled.

The second photoelectric conversion layer 17 may be interposed between the first photoelectric conversion layer 12 and the counter electrode 13. In such a case, the second photoelectric conversion layer 17 absorbs visible light and the effect of visible light in photoelectric conversion of the first photoelectric conversion layer 12 is reduced The image quality of the first infrared image obtained may thus be improved. Since the pixel 10b includes the second photoelectric conversion layer 17 having a spectral sensitivity to visible light, the first photoelectric conversion layer 12 may not necessarily have a spectral sensitivity to visible light. The pixel 10b may include the hole transport layer 15 and the hole blocking layer 16 as the pixel 10a does.

### Second Embodiment

A biometric authentication system 3 of a second embodiment is described below. The following discussion focuses on the difference from the first embodiment and the modification of the first embodiment and common parts thereof are briefly described or not described at all.

### Configuration

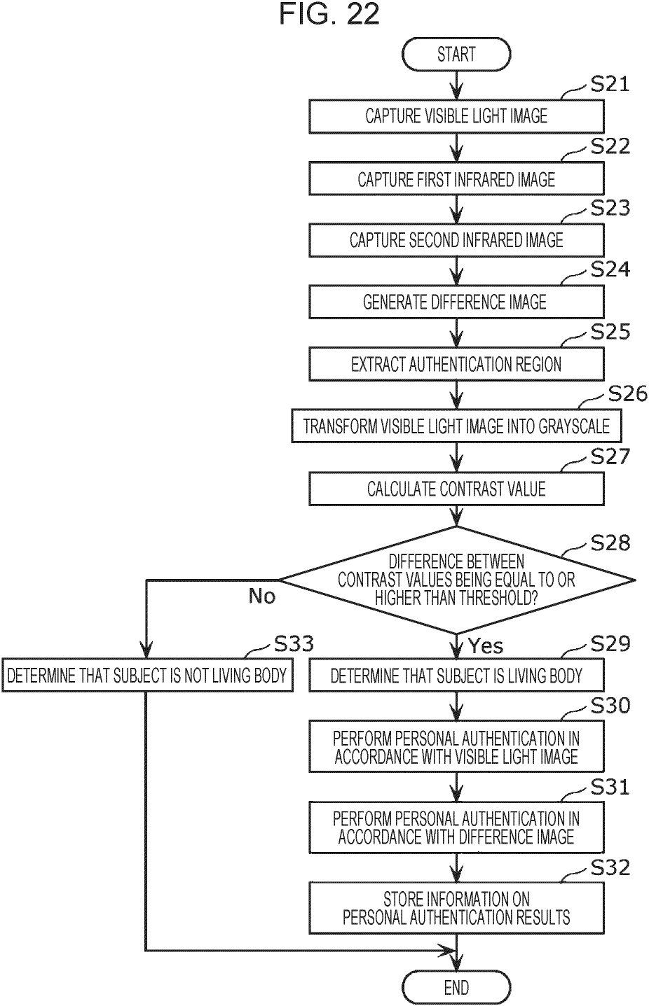

The configuration of the biometric authentication system 3 of the second embodiment is described below. FIG. 21 is a block diagram illustrating a functional configuration of the biometric authentication system 3 of the second embodiment.

Referring to FIG. 21, the biometric authentication system 3 of the second embodiment is different from the biometric authentication system 1 of the first embodiment in that the biometric authentication system 3 includes a processor 102 and an imager 302, in place of the processor 100 and the imager 300, and a second light illuminator 420.

The processor 102 includes, besides the structure of the processor 100, a third image capturer 113 included in the memory 600.

The third image capturer 113 captures a second infrared image of the subject. The third image capturer 113 temporarily stores the second infrared image of the subject. The second infrared image is imaged by picking up light that is reflected from the subject irradiated with infrared light and includes the wavelength region including a second wavelength different from the first wavelength. The third image capturer 113 captures the second infrared image from the imager 302, specifically, from a fourth imaging device 314 in the imager 302.

The determiner 120 in the biometric authentication system 3 determines whether the subject is a living body, in accordance with the visible light image captured by the first image capturer 111, the first infrared image captured by the second image capturer 112, and the second infrared image captured by the third image capturer 113.

The imager 302 includes, besides the structure of the imager 300, the fourth imaging device 314.

The fourth imaging device 314 images the second infrared image of the subject. The fourth imaging device 314 receives light that is reflected from the subject irradiated with infrared light and includes the wavelength region including the second wavelength. The fourth imaging device 314 generates the second infrared image by imaging the incident reflected light. The fourth imaging device 314 outputs the generated second infrared image. The fourth imaging device 314 is identical in structure to the second imaging device 312 except that the wavelength having a spectral sensitivity is different. The reason why the second wavelength is selected is identical to the reason why the first wavelength is selected. For example, a wavelength different in water absorption coefficient from the first wavelength is selected as the second wavelength in the same way as the first wavelength. The fourth imaging device 314 may be an imaging device that operates in a global shutter method in which exposure periods of multiple pixels are unified.

The second light illuminator 420 irradiates the subject with infrared light, within the wavelength region including the second wavelength, as the irradiation light. The fourth imaging device 314 images the light that is reflected from the subject irradiated with infrared light from the second light illuminator 420. The second light illuminator 420 emits infrared light having an emission peak on or close to the second wavelength. The second light illuminator 420 is identical in structure to the first light illuminator 410 except that the wavelength of the irradiation light is different.

The biometric authentication system 3 may include a single light illuminator that has the functions of the first light illuminator 410 and the second light illuminator 420. In such a case, the image illuminator irradiates the subject with infrared light within the wavelength range including the first wavelength and the second wavelength. The light illuminator includes a first light emitter, such as a light emitting diode (LED), having an emission peak on or close to the first wavelength and a second light emitter, such as an LED, having an emission peak on or close to the second wavelength, and causes the first light emitter and the second light emitter to alternately light by selectively switching between the first light emitter and the second light emitter. The first light emitters and the second light emitters may be arranged in a zigzag fashion. The light illuminator may include a halogen light source that has a broad light spectrum within the wavelength range of infrared light. Since the unitary light illuminator irradiates the subject in a coaxial manner with infrared light within the wavelength range including the first wavelength and infrared light within the wavelength range including the second wavelength, a difference caused by the shadow of the irradiation light may be reduced.

The timing controller 500 in the biometric authentication system 3 controls the imaging timing of the imager 302, the irradiation timing of the first light illuminator 410, and the irradiation timing of the second light illuminator 420. For example, the timing controller 500 outputs the first synchronization signal to the second imaging device 312 and the first light illuminator 410, and outputs a second synchronization signal different from the first synchronization signal to the fourth imaging device 314 and the second light illuminator 420. The second imaging device 312 images the first infrared image at the timing responsive to the first synchronization signal. The first light illuminator 410 irradiates the subject with infrared light at the timing responsive to the first synchronization signal. The fourth imaging device 314 images the second infrared image at a timing responsive to the second synchronization signal. The second light illuminator 420 irradiates the subject with infrared light at the timing responsive to the second synchronization signal. In this way, the timing controller 500 causes the second imaging device 312 to image the first infrared image while the first light illuminator 410 irradiates the subject with infrared light and causes the fourth imaging device 314 to image the second infrared image while the second light illuminator 420 irradiates the subject with infrared light. The timing controller 500 outputs the first synchronization signal and the second synchronization signal at different timings such that the infrared irradiation time of the first light illuminator 410 and the infrared irradiation time of the second light illuminator 420 do not conflict. In this way, the first infrared image and the second infrared image are imaged with the effect of infrared light of an unintended wavelength minimized.

### Process

The process performed by the biometric authentication system 3 is described below. FIG. 22 is a flowchart illustrating a process example of the biometric authentication system 3 of the second embodiment. The process in FIG. 22 is a process method that is performed by the processor 102 in the biometric authentication system 3.

The first image capturer 111 captures the visible light image (step S21). The second image capturer 112 captures the first infrared image (step S22). The operations in steps S21 and S22 are respectively identical to the operations in steps S1 and S2.

The third image capturer 113 captures the second infrared image (step S23). The second light illuminator 420 irradiates the subject with infrared light within the wavelength range including the second wavelength. The fourth imaging device 314 images the second infrared image by acquiring light that is reflected from the subject irradiated with infrared light from the second light illuminator 420 and includes the wavelength region including the second wavelength. In this case, the timing controller 500 outputs the second synchronization signal to the fourth imaging device 314 and the second light illuminator 420 and the fourth imaging device 314 images the second infrared image in synchronization with the infrared irradiation of the second light illuminator 420. The third image capturer 113 captures the second infrared image imaged by the fourth imaging device 314.

The fourth imaging device 314 may image multiple second infrared images. For example, the fourth imaging device 314 images two second infrared images when the second light illuminator 420 under the control of the timing controller 500 emits infrared light and when the second light illuminator 420 under the control of the timing controller 500 does not emit infrared light. The determiner 120 or the like determines a difference between the two second infrared images, thereby generating an image with the ambient light offset. The resulting image may thus be used in the impersonation determination and the personal authentication.

The determiner 120 generates a difference infrared image from the first infrared image and the second infrared image (step S24). For example, the determiner 120 generates the difference infrared image by calculating a difference between the first infrared image and the second infrared image or calculating a ratio of luminance values.

If the first wavelength is a missing wavelength of the sunlight and happens to be 1,400 nm likely to be absorbed by the water component, and the second wavelength is 1,550 nm, it may be difficult to determine whether the first infrared image of the subject is darkened by the absorption by the water component or by the shadow of the irradiation light. The generation of the difference infrared image between the first infrared image and the second infrared image may remove the effect attributed to the darkened image caused by the shadow of the irradiation light. The accuracy of the impersonation determination based on the principle of the absorption by the water component may be increased.

From each of the visible light image captured by the first image capturer 111 and the generated difference infrared image, the determiner 120 extracts an authentication region serving as a region where the subject is depicted (step S25). The extraction of the authentication region is identical to the operation in step S3.

The determiner 120 transforms to grayscale the visible light image from which the authentication region is extracted in step S25 (step S26). The determiner 120 may also transform to grayscale the difference infrared image from which the authentication region is extracted. In such a case, the visible light image from which the authentication region is extracted and the difference infrared image from which the authentication region is extracted may be grayscale-transformed on the same level quantization (for example, 16-level quantization). In the following discussion, the visible light image and the difference infrared image having undergone the operations from step S21 through step S26 are respectively referred to as a determination visible light image and a determination difference infrared image.

The determiner 120 calculates contrast values from the determination visible light image and the determination difference infrared image (step S27). The calculation of the contrast value by the determiner 120 in step S27 is identical to the operation in step S5 except that the determination difference infrared image is used in step S27 in place of the determination first infrared image.

The determiner 120 determines whether a difference between the contrast values of the determination visible light image and the determination difference infrared image calculated in step S27 is higher than or equal to a threshold (step S28). If the difference between the contrast values of the determination visible light image and the determination difference infrared image is higher than or equal to the threshold (yes path in step S28), the determiner 120 determines that the subject is a living body and outputs the determination results to the first authenticator 131, the second authenticator 132 and the outside (step S29). If the difference between the contrast values of the determination visible light image and the determination difference infrared image calculated in step S27 is lower than the threshold (no path in step S28), the determiner 120 determines that the subject is not a living body, and outputs the determination results to the first authenticator 131, the second authenticator 132, and the outside (step S33). The operations in steps S28, S29, and S33 are respectively identical to the operations in steps S6, S7, and S11 except that the determination difference infrared image is used in steps S28, S29, and S33 in place of the determination first infrared image. The processor 102 ends the process after step S33 in the same way as after step S11.

After receiving the determination results from the determiner 120 having determined in step S29 that the subject is the living body, the first authenticator 131 performs the personal authentication on the subject in accordance with the visible light image and outputs the results of the personal authentication to the outside (step S30). After receiving the determination results from the determiner 120 having determined in step S29 that the subject is the living body, the second authenticator 132 performs the personal authentication on the subject in accordance with the difference infrared image and outputs the results of the personal authentication to the outside (step S31). The second authenticator 132 acquires the difference infrared image from the determiner 120. The operations in steps S30 and S31 are respectively identical the operations in steps S8 and S9 except that the difference infrared image is used in steps S30 and S31 in place of the first infrared image.

The information constructor 140 stores, in an associated form on the storage 200, information on the results of the personal authentication performed by the first authenticator 131 and information on the results of the personal authentication performed by the second authenticator 132 (step S32). The information constructor 140 also registers, in an associated form on the personal authentication database on the storage 200, the visible light image and the difference infrared image, authenticated through the personal authentication. The information constructor 140 may store, in an associated form on the personal authentication database of the storage 200, the first infrared image and the second infrared image prior to the generation of the difference infrared image used in the personal authentication and the visible light image authenticated through the personal authentication. Subsequent to step S32, the processor 102 in the biometric authentication system 3 ends the process.

In the same way as the first embodiment, the first authenticator 131 and the second authenticator 132 may perform the personal authentication regardless of the determination results of the determiner 120. The determiner 120 may perform the impersonation determination without generating the difference infrared image. For example, the determiner 120 compares the contrast values calculated from the visible light image, the first infrared image, and the second infrared image to determine whether the subject is a living body.

### Modification

A biometric authentication system 4 as a modification of the second embodiment is described below. The following discussion focuses on the difference from the first embodiment, the modification of the first embodiment, and the second embodiment and common parts thereof are briefly described or not described at all.

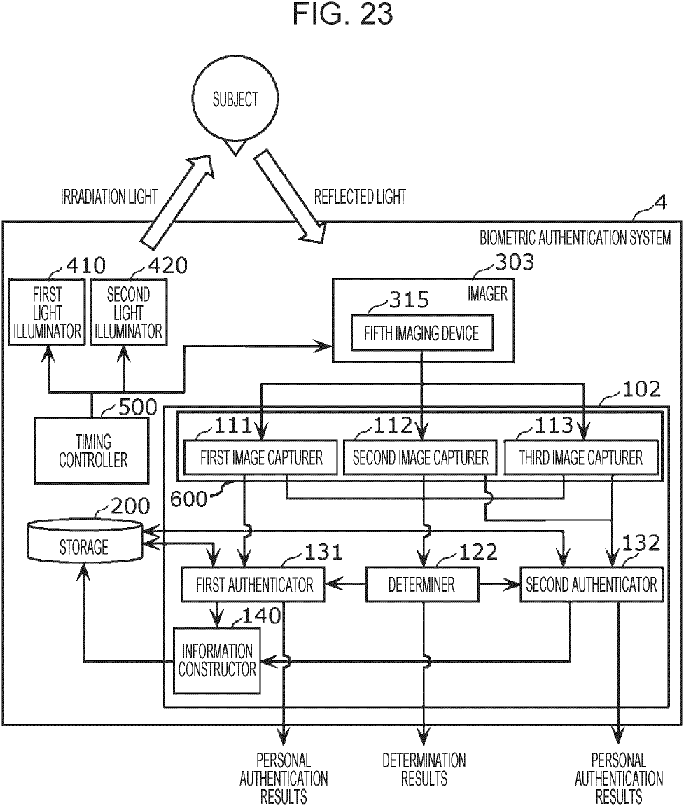

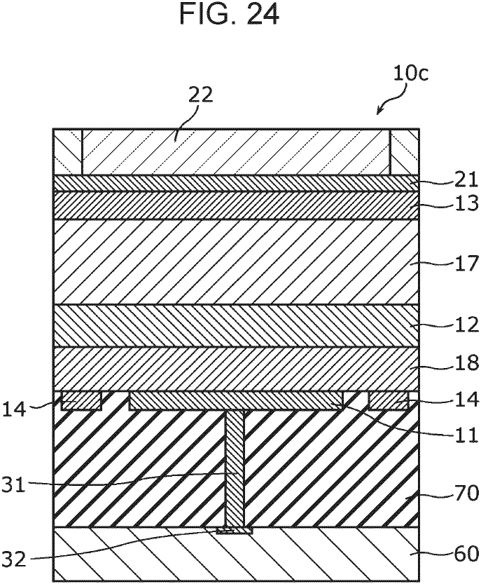

FIG. 23 is a block diagram illustrating a functional configuration of the biometric authentication system 4 according to the modification of the second embodiment;

Referring to FIG. 23, the biometric authentication system 4 as the modification of the second embodiment is different from the biometric authentication system 3 in that the biometric authentication system 4 includes an imager 303 in place of the imager 302.

The imager 303 includes a fifth imaging device 315 that images the visible light image, the first infrared image, and the second infrared image. As described below, for example, the fifth imaging device 315 may be implemented by an imaging device that includes a photoelectric conversion layer having a spectral sensitivity to visible light and infrared light in two wavelength regions. The fifth imaging device 315 may be an InGaAs camera that has a spectral sensitivity to visible light and infrared light. Since the imager 303 including the fifth imaging device 315 as a single imaging device is able to image all of the visible light image, the first infrared image, and the second infrared image, the biometric authentication system 4 may thus be down-sized. Since the fifth imaging device 315 is able to image in a coaxial fashion the visible light image, the first infrared image, and the second infrared image, the effect of parallax may be controlled by the visible light image, the first infrared image, and the second infrared image. The authentication accuracy of the biometric authentication system 4 may thus be increased. The fifth imaging device 315 may be an imaging device that operates in a global shutter method in which exposure periods of multiple pixels are unified.

The first image capturer 111 in the biometric authentication system 4 captures the visible light image from the fifth imaging device 315, the second image capturer 112 captures the first infrared image from the fifth imaging device 315, and the third image capturer 113 captures the second infrared image from the fifth imaging device 315.

The timing controller 500 in the biometric authentication system 4 controls the imaging timing of the imager 303, the irradiation timing of the first light illuminator 410, and the irradiation timing of the second light illuminator 420. The timing controller 500 outputs the first synchronization signal to the fifth imaging device 315 and the first light illuminator 410, and outputs the second synchronization signal to the fifth imaging device 315 and the second light illuminator 420. The fifth imaging device 315 images the first infrared image at the timing responsive to the first synchronization signal and images the second infrared image at the timing responsive to the second synchronization signal. In this way, the timing controller 500 causes the fifth imaging device 315 to image the first infrared image while the first light illuminator 410 irradiates the subject with infrared light and causes the fifth imaging device 315 to image the second infrared image while the second light illuminator 420 irradiates the subject with infrared light.

The biometric authentication system 4 operates in the same way as the biometric authentication system 3 except that the first image capturer 111, the second image capturer 112, and the third image capturer 113 respectively capture the visible light image, the first infrared image, and the second infrared image from the fifth imaging device 315 in the biometric authentication system 4.

The configuration of the fifth imaging device 315 is specifically described below.

The fifth imaging device 315 includes multiple pixels 10c in place of the pixels 10 in the third imaging device 313 illustrated in FIG. 15. The imaging region R1 includes the pixels 10c that include optical filters 22 different from each other in transmission wavelength range and respectively used for infrared light within a wavelength range including the first wavelength, infrared light within a wavelength range including the second wavelength, blue light, green light, and red light. In this way, image signals respectively responding to the infrared light within the wavelength range including the first wavelength, the infrared light within the wavelength range including the second wavelength, blue light, green light, and red light are separately read. The fifth imaging device 315 generates the visible light image, the first infrared image, and the second infrared image using these image signals.

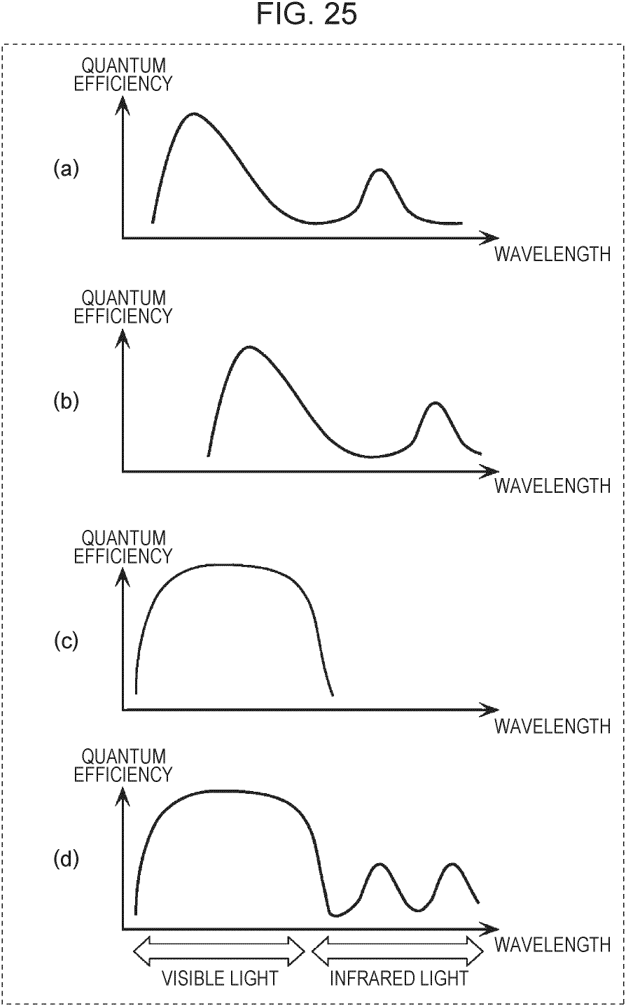

FIG. 24 is a schematic cross-sectional view illustrating a cross-sectional structure of a pixel 10c of the fifth imaging device 315 according to the modification of the second embodiment. The pixels 10c are identical to each other in structure except that transmission wavelength of each optical filter 22 is different. Some of the pixels 10c may be different from the rest of the pixels 10c not only in the optical filter 22 but also in another portion.

Referring to FIG. 24, the pixel 10c includes, besides the structure of the pixel 10b, a third photoelectric conversion layer 18. In other words, the pixel 10c includes, besides the structure of the pixel 10, the second photoelectric conversion layer 17 and the third photoelectric conversion layer 18.

In the pixel 10c, the second photoelectric conversion layer 17 is interposed between the first photoelectric conversion layer 12 and the counter electrode 13. The third photoelectric conversion layer 18 is interposed between the first photoelectric conversion layer 12 and the pixel electrode 11. As long as the first photoelectric conversion layer 12, the second photoelectric conversion layer 17, and the third photoelectric conversion layer 18 are interposed between the pixel electrode 11 and the counter electrode 13, the first photoelectric conversion layer 12, the second photoelectric conversion layer 17, and the third photoelectric conversion layer 18 may be laminated in any lamination order.

The third photoelectric conversion layer 18 absorbs infrared light within the wavelength range of visible light and the second wavelength. Specifically, the third photoelectric conversion layer 18 has a spectral sensitivity to the second wavelength of infrared light and the wavelength range of visible light. For example, the third photoelectric conversion layer 18 has a spectral sensitivity peak on the second wavelength.

The third photoelectric conversion layer 18 absorbs light within the wavelength range of infrared light including the second wavelength and the wavelength range of visible light and contains a donor material generating hole-electron pairs. The donor material contained in the third photoelectric conversion layer 18 may be selected from the group of materials cited as the donor materials contained in the first photoelectric conversion layer 12. For example, the third photoelectric conversion layer 18 may contain semiconductor quantum dots as the donor material.

FIG. 25 schematically illustrates an example of spectral sensitivity curves of the pixel 10c. Part (a) of FIG. 25 illustrates the relationship between the external quantum efficiency of the first photoelectric conversion layer 12 and the wavelength of light. Part (b) of FIG. 25 illustrates the relationship between the external quantum efficiency of the third photoelectric conversion layer 18 and the wavelength of light. Part (c) of FIG. 25 illustrates the relationship between the external quantum efficiency of the second photoelectric conversion layer 17 and the wavelength of light. Part (d) of FIG. 25 illustrates the relationship between the external quantum efficiency and the wavelength of light of all the pixels 10c when the sensitivities of the first photoelectric conversion layer 12, the second photoelectric conversion layer 17, and the third photoelectric conversion layer 18 are combined.

Referring to parts (a) and (b) of FIG. 25, each of the first photoelectric conversion layer 12 and the third photoelectric conversion layer 18 has a spectral sensitivity to the wavelength range of visible light and infrared light. A spectral sensitivity peak of the first photoelectric conversion layer 12 and a spectral sensitivity peak of the third photoelectric conversion layer 18 is different within the wavelength range of infrared light. Referring to part (c) of FIG. 25, the second photoelectric conversion layer 17 has a spectral sensitivity to the wavelength range of visible light wider than the wavelength range of visible light to which each of the first photoelectric conversion layer 12 and the third photoelectric conversion layer 18 have the spectral sensitivity. For this reason, as illustrated in part (d) of FIG. 25, all the pixels 10c has two spectral sensitivity peaks within the wavelength range of infrared light and also has a spectral sensitivity within the whole wavelength range of visible light. Since the pixels 10c have such a spectral sensitivity property, the fifth imaging device 315 may image all of the visible light image, the first infrared image and the second infrared image.

Since the pixel 10c includes the second photoelectric conversion layer 17 having a spectral sensitivity to visible light, at least one of the first photoelectric conversion layer 12 or the third photoelectric conversion layer 18 may not necessarily have a spectral sensitivity to visible light. As long as the spectral sensitivity curve illustrated in part (d) of FIG. 25 is provided, the pixel 10c may not necessarily include three photoelectric conversion layers. The pixel 10c may be implemented using one or two photoelectric conversion layers depending on a material selected for the photoelectric conversion layer. The pixel 10c may include the hole transport layer 15 and the hole blocking layer 16 in the same way as the pixel 10a.

### OTHER EMBODIMENTS

The biometric authentication systems of the embodiments of the disclosure have been described. The disclosure is not limited to the embodiments and the modifications thereof.

According to the embodiments and the modifications thereof, the determiner compares the contrast values to determine whether the subject is a living body. The disclosure is not limited to this method. The determiner may determine whether the subject is a living body, by performing the comparison in accordance with the difference between luminance values of adjacent pixels or in accordance with a difference in a balance of luminance values, such as histograms of the luminance values.

According to the embodiments and the modification thereof, the biometric authentication system incudes multiple apparatuses. Alternatively, the biometric authentication system may be implemented using a single apparatus. If the biometric authentication system is implemented by multiple apparatuses, elements included in the biometric authentication system described may be distributed among the apparatuses in any way.

The biometric authentication system may not necessarily include all the elements described with reference to the embodiments and the modifications thereof and may include only elements intended to perform a desired operation. For example, the biometric authentication system may be implemented by a biometric authentication apparatus having the functions of the first image capturer, the second image capturer, and the determiner in the processor.

The biometric authentication system may include a communication unit and at least one of the storage, the imager, the first light illuminator, the second light illuminator, or the timing controller may be an external device, such as a smart phone or a specialized device carried by a user. The impersonation determination and the personal authentication may be performed by the biometric authentication system that communicates with the external device via the communication unit.

The biometric authentication system may not necessarily include the first light illuminator and the second light illuminator and use the sunlight or the ambient light as the irradiation light.

According to the embodiments, an operation to be performed by a specific processor may be performed by another processor. The order of operations may be modified or one operation may be performed in parallel with another operation.

According to the embodiments, each element may be implemented by a software program appropriate for the element. The element may be implemented by a program executing part, such as a CPU or a processor, that reads a software program from a hard disk or a semiconductor memory, and executes the read software program.

The elements may be implemented by a hardware unit. The elements may be circuitry (or an integrated circuit). The circuitry may be a unitary circuit or include several circuits. The circuits may be a general-purpose circuit or a specialized circuit.

Generic or specific form of the disclosure may be implemented by a system, an apparatus, a method, an integrated circuit, a computer program, or a recording medium, such as a computer-readable compact disc read-only memory (CD-ROM). The generic or specific form of the disclosure may be implemented by any combination of the system, the apparatus, the method, the integrated circuit, the computer program, and the recording medium.

The disclosure may be implemented as the biometric authentication system according to the embodiments, a program causing a computer to execute the biometric authentication method to be performed by the processor, or a computer-readable non-transitory recording medium having stored the program.

Without departing from the spirit of the disclosure, a variety of changes conceived by those skilled in the art in the embodiments and modifications may fall within the scope of the disclosure and another embodiment constructed by a subset of the elements in the embodiments and modification may also fall within the scope of the disclosure.

The biometric authentication system of the disclosure may be applicable to a variety of biometric authentication systems for mobile, medical, monitoring, vehicular, robotic, financial, or electronic-payment application.

What is claimed is:

1. A biometric authentication system comprising:
a first image capturer that captures a visible light image that is imaged by picking up first light reflected from a skin portion of a subject that is irradiated with visible light;
a second image capturer that captures a first infrared image that is imaged by picking up second light that is reflected from the skin portion irradiated with first infrared light and that has a wavelength region including a first wavelength; and
a determiner that determines, in accordance with a result of comparing the visible light image with the first infrared image, whether the subject is a living body and outputs a determination result.

2. The biometric authentication system according to claim 1, further comprising a first authenticator that performs first personal authentication on the subject in accordance with the visible light image and that outputs a result of the first personal authentication.

3. The biometric authentication system according to claim 2, wherein if the determiner determines that the subject is not the living body, the first authenticator does not perform the first personal authentication on the subject.

4. The biometric authentication system according to claim 2, further comprising a second authenticator that performs second personal authentication on the subject in accordance with the first infrared image and that outputs a result of the second personal authentication.

5. The biometric authentication system according to claim 4, further comprising:
a storage that stores information used to perform the first personal authentication and the second personal authentication; and
an information constructor that causes the storage to store information on the result of the first personal authentication and information on the result of the second personal authentication in an associated form.

6. The biometric authentication system according to claim 1, wherein the determiner compares a contrast value based on the visible light image with a contrast value based on the first infrared image to determine whether the subject is the living body.

7. The biometric authentication system according to claim 1, further comprising an imager that includes a first imaging device imaging the visible light image and a second imaging device imaging the first infrared image, wherein
the first image capturer captures the visible light image from the first imaging device, and
the second image capturer captures the first infrared image from the second imaging device.

8. The biometric authentication system according to claim 1, further comprising an imager that includes a third imaging device imaging the visible light image and the first infrared image, wherein
the first image capturer captures the visible light image from the third imaging device, and
the second image capturer captures the first infrared image from the third imaging device.

9. The biometric authentication system according to claim 8, wherein the third imaging device includes a first photoelectric conversion layer having a spectral sensitivity to a wavelength range of the visible light and the first wavelength.

10. The biometric authentication system according to claim 9, wherein the third imaging device includes a second photoelectric conversion layer having a spectral sensitivity to an entire wavelength range of visible light.

11. The biometric authentication system according to claim 7, further comprising a light illuminator that irradiates the subject with the first infrared light.

12. The biometric authentication system according to claim 11, further comprising a timing controller that controls an imaging timing of the imager and an irradiation timing of the light illuminator.

13. The biometric authentication system according to claim 1, further comprising a third image capturer that captures a second infrared image that is imaged by picking up third light that is reflected from the skin portion irradiated with second infrared light and that has a wavelength region including a second wavelength different from the first wavelength,
wherein the determiner determines, in accordance with the visible light image, the first infrared image, and the second infrared image, whether the subject is the living body.

14. The biometric authentication system according to claim 13, wherein the determiner generates a difference infrared image between the first infrared image and the second infrared image and determines, in accordance with the difference infrared image and the visible light image, whether the subject is the living body.

15. The biometric authentication system according to claim 1, wherein the first wavelength is shorter than or equal to 1,100 nm.

16. The biometric authentication system according to claim 1, wherein the first wavelength is longer than or equal to 1,200 nm.

17. The biometric authentication system according to claim 1, wherein the first wavelength is longer than or equal to 1,350 nm and shorter than or equal to 1,450 nm.

18. The biometric authentication system according to claim 1, wherein the subject is a human face.

19. A biometric authentication method comprising:
capturing a visible light image that is imaged by picking up first light reflected from a skin portion of a subject that is irradiated with visible light;
capturing a first infrared image that is imaged by picking up second light that is reflected from the skin portion irradiated with first infrared light and that has a wavelength region including a first wavelength; and
determining, in accordance with a result of comparing the visible light image with the first infrared image, whether the subject is a living body and outputting a determination result.
